SEQUENCE LISTING (85 sequences)

SEQ ID NO: 1 - protein, 582 aa, unknown
  bacterial
MRQLKKWMLAAILICGISVFTACSSNDDNSSPVPQPDLNLAEKIVGKWMVAELNGEACPTNLKTVVTFDSPTKAYGSLSDFYSKSWNDEVEADVKIDGNKMLITAKEDDHTTHVLDVTVSSITDKDMVLSSNWSVLVDGKEVHHEAYEEERWERVKNDYESAFYGLWEGKVTRDLGDETNDELHRWECMAAGTYAFYDKVGDKWVEAPHYLADYFVDGTLLCTRWQDTKDSEELREWWEIESIKDDVVKATALRVREDGSTYTASFQMTRVQPEIIDYSDKVNWLAFPEITKDVDAIYIYSTSYVESSFDDGASNYVPIDNPEMIMFANGEYETNATLFEESCNVFAPYYRQAGMRYANEIAKKTGNIDAALAGLSYTDIKASLDYYFENCNNGRPFIIAGHSQGSAMVRYVLKNYFSEHLDYYQRMVAAYPIGFAITKDDLEAYPHLKFATGESDTGVIISYNTEGPKNVEENARNVAVLPGAISINPLNWKLDETYASASENKGSLVQNKETGAREFVDLGVDAQINLARGVIVTKTTAPVTDGKEFFGPASFHENDYSFFYKNLQENVAKRIAAYKNNN

SEQ ID NO: 2 - protein, 580 aa, unknown
  bacterial
MRQLKKWMLAAILICGTSVFMACSSNEDNPAPQPDINLAEKIVGKWMVAELNGEACPTNLKTVVTFDSPTKAYGSLSDFYSKSWNDEVEADVKINGNKILITAKEDDNTTHVLDVTVSSITDTDMVLSSDWTVLVDGKEVYQEAYEEERWERVKNDYESAFYGLWEGKVTRDLGDETNDELHRWECMAAGTYAFYDKVGDKWVEAPHYLADYFVDGTLLCTRWQDTKDSEELREWWEIESIKDDVVKATALRVREDGSTYTASFQMTRVQPETIDYSDKANWLAFPEITKDVDAIYIYSTSYVESSFDDGASNYVPIDNPEMIMFANGEYETNATLFEESCNVFAPYYRQAGMRYANEIAKKTGNIDTALAGLSYTDIKASLDYYFENCNNGRPFIIAGHSQGSAMVRYVLKNYFSEHLDYYQRMVAAYPIGFAITKDDLEAYPHLKFATGESDTGVIISYNTEGPKNVEENARNVAVLPGAISINPLNWKLDETYASASENKGSLVQNKETGAREFVDLGVDAQINLARGVIVTNTTAPVTEGKEFFGPASFHENDYSFFYKNLQENVAKRIAAYKNNN

SEQ ID NO: 3 - protein, 352 aa, unknown
  bacterial
MKRQNFLVLLASAMLTAMTVASCKSSNKQTAGADAQDSIPEVLKDVAGIEKFQTKLDYSKAENWVERPTEATKPVDVFFIYPTVTGFRDPVQICEVTDSEMVAGAKFVRQVQTSVFEESCNIFMPFYRQISMPKPGTDYRFVTNYLSGFDATDALDYFLTHLNQGRPFILAGHSQGSSTLLNLLENYMTKHPEHLSRMVAAYPIGFAVTKDFLERTGLKFAEGAIDTGVIVSWNTEGVGNKDAKNATLAPDGLSINPINWKRDDTYAPVKDNLGSLDETTGKVIVPGIADARVDTVRGSVVVTTAAAKPFAISEKEAVPLFGPECYHRNDYGFFYNNLKQNIADRIRAFVEK

SEQ ID NO: 4 - protein, 361 aa, unknown
  bacterial
MQTTHRIIKMKRQNSLVLLASVMLAAMSLASCKSSNKPATDASAQDSVPEVLKDVAGIEKFQTKLDYSKTENWVERPAEAKKPVDVFFIYPTVTGFRDPVQICEVTDSEMVAGAQMVRRVQTSVFEESCNIYMPYYRQISMPKPGTDYRFVTNYLSGFDATDALDYFLTHLNQGRPFILAGHSQGSSTLLNLLENYMTKHPEHLSRMVAAYPIGFAVTKDFLERTGLKFAEGATDTGVIVSWNTEGVGNKDAKNATLAPDGLSINPINWKRDDTYASAKDNLGSLDEMTGRVIVPGIVDARVDTVRGSVVVTTAAAKPFAISEKEAVPLFGPECYHRNDYGFFYNNLKQNIADRIRAFVEK

SEQ ID NO: 5 - protein, 352 aa, unknown
  bacterial
MIMKRQNILVLLASAMLVAMNLASCKMSNKPAADVATEDSIPEVLKDVAGLEKYQTKLDYSKDECWLEKPAEATKPVDVFYIYPTVTGFRDPVQICEITDSELVAGAKMVRQIQTSVFDESCNVFMPYYRQISMPKPGTDYSSVTNYLSGFDATDALDYFLNNLNQGRPFILAGHSQGASTLINLLENYMTKHPEALKRMVAAYPVGFAVTKDFLARTGLKFAEGATDTGVIVSWNTEGAANKDAKNVVIAPGGISINPINWKRDDTYAPVSDNLGSLDITGKLVTPGFADARVDTVRGSVIVTTADAATYTIPADGAPLFGPESYHLHDYGFFFNNFKQNVADRVKAFLEK

SEQ ID NO: 6 - protein, 281 aa, unknown
  bacterial
MMRSLKVLICVICVICGFIAATPDKTTTIFVVGDSTAANKDTTGGKVERGWGMMLQQCFDADFIVVDNHAVNGRSSKSFLDEGRWDKVLEKIQPGDYVLIQFGHNDEKAQPARHTDPGSTFDYNLARYVRETREMGGIPVLMNAVARRNFFIMPPEVADDEQLRNTIFSDSEHLVEGDSLNDTHGLYRVAPRDVARRMNCHFIDANRITEDLEKRLGREGSKKLHMWFRPGEEPSLPQGKQDNTHYNIYGAQVVANLLADALCEEIPVLKPYRVKGVRNAK

SEQ ID NO: 7 - protein, 525 aa, unknown
  bacterial
MMRFYMEQYVENQKIIDGIYDQALAVKCINGTFVGKKAENIISYKGIPFVGQQPVGNLRWKAPVDVTPDDGVYEAYYYGKAPVQEVGDPAAEYGTGEDCLYLNVWKADEAAAKKPVIVWIYGGAFDVGGATDPMYDLTNFLNENPDVIVVTLNYRINVFGFFHLSHLSDGKDYPDAQNLGLLDQLMALKWVHENIAAFGGDPDNVTIWGESAGAGSCTLLPLIKGSHQYFKRVIAQSGAPAQTRSAEQAIACTNSLMEKLGCKTVADLLKISAEDIMKTWAPILGLYSALGIRTMPERDGIYLPLDPWEAYANGAAKDIEFLQGCDKDEMNTFMGALGEEAWNAWAKERTAEKLAQLTDQEKALVESYLNSIKGENYEATSNLYSQMMFIAPQIRLSDEQFKAGGKSYTYYYRVESSQPYIKAGHASELPVVFAHPEIQEFTGRAYDETFSKTMRKMWIQFAKTGNPSLSAAESPDGKAKEWPLYDMKDRQVMVLDEFDIHTAKESELKIVDWERTYFLTNYYMP

SEQ ID NO: 8 - protein, 527 aa, unknown
  bacterial
MDAQEIRDALHGMYGGENKQITDGNYDKSLAVKCINGTFVGRKNDNIIVYRGIPYVGKQPVGDLRWKAPVDAVPNEGVYEAYYNAKSGYGNPSFEVGSLYYLDEDSLYLNVFKADDATTDKKPVMVWIHGGAFESGGTIDPMFDCINFVKENPEVVVVTIAYRLGVMGFLHLSHLPDGKDYQDSQNLGLLDQKMALKWVHENIAGFGGDPDNVTIFGESAGAASCTLQPLVPGSQKYFKRLIAESGSVNQTRSPEEGIEGTNKLMEALGCKTVADLLKVDGDKLLETATAVNFAHQMPERDGKYLPTDTFEAYANGAAKDLEILVGCNKDEMNFFVSSFGKEGWDNWIAARKQEKYDKLPTDEKALVDSYMKDVKGDYFQPDSSLFSQSWFIVPTIKLSECQTMGGGKAYTYYFTPESPDPIMKCGHAIELSVVFNHPEMSADTGREFDATFCKTMRKMWVQFAKTGNPSLSADLSPDGKAKEWPLYDLDNKRVMVLDEFDIHPAKESEVKIVDWERTYFLTKYYML

SEQ ID NO: 9 - protein, 526 aa, unknown
  bacterial
MDAQEIRDALHGMYGGENKPITDGNYDQSLAVKCINGTFVGRKNDNIIVYRGIPYVGKQPVGDLRWKAPVDAVPNEGVYEAYYNAKSGYGNPSFEVGSLYYLSEDSLYLNVFKADDATTDKKPVMVWIHGGAFESGGTIDPMFDCINFVKENPEVVVVTIAYRLGVMGFLHLSHLPDGKDYQDSQNLGLLDQLKALKWVHENIAGFGGDPDNVTIFGESAGAASCTLQPLLPGSHQYFKRLIAESGSVNQTRSPEEAIECTNKLMDVLSCKTVADLLKVSGDKLLEASTVNFVHQMPERDGKYLPTDTFAAYANGAAKDLQILVGCNKDEMNFFVSSFGKEGWDNWIAARKQEKFDQLPADEKALVDSYMKDVKGDYFQPDSSLFSQSWFIVPTIKLSECQTMGGGKAYTYYFTPESPDPIMKCGHAIELSVVFNHPEMSADTGREFDATFCKTVRKMWVQFAKTGNPSLSADLSPDGKAKEWPLYDLDNKRVMVLDEFDIHPAKESEVKIVDWERTYFLTKYYML

SEQ ID NO: 10 - protein, 520 aa, unknown
  bacterial
MEQYVKNQKITDGHYDKSLAVKCINGTFVGKKAENVISYKGIPFVGQQPVGNLRWKAPVDIVPDDSIYEAYYYGKAPIQEPGDPAAEYGTSEDCLYLNVWKADEAAAKKPVIVWIYGGAFDVGGATDPMYNLTNFLNENPDVIVVTISYRINVFGFFHLSHLSDGKDYPDAQNLGLLDQLMALKWVHENIAAFGGDPDNVTIWGESAGAGSCTLLPLIKGSHQYFKRVIAQSGAPAQTRSAEQAIACTNSLMEKLGCKTVADLLKISAEDIMKTWAPILGLYSALGIRTMPERDGIYLPLDPWEAYANGAAKDIEFLQGCDKDEMNTFMGALGEEAWNAWAKERTAEKFAQLTDKEKALVESYLNDIQGESYEPTVNLFSQMMFIAPQIRLSEEHTKSGGKSYTFYYRVESSQPYIKAGHASELPVVFAHPEIQEFTGRAYDETFSKTMRKMWVQFAKTGNPSLSAAESPDGKAKEWPLYDMKDRQVMVLDEFDIHTAKESELKIVDWERTYFLTNYYMP

SEQ ID NO: 11 - protein, 519 aa, unknown
  bacterial
MSEKEEQLRAQYSENQKITGGNYDKSLAVKCINGIFVGKKTENIISYKGIPFVGQQPIGELRWKAPVEYSSNDGVYEAYHFEKMSVQDLSITDHQGEDCLYLNVYKADDASEVKKPVMVWIHGGAFVAGGTGIELFDCTELVKGNPDVIVVTVAYRLGVLGFFHLSHLADGKDYPDAQNLGLLDQKMALKWVHENIAGFGGDPDNVTIWGESAGAGSVTMQPLIKGSQQYFKKVIAQSGAPSQMNSVEESISTTNDLMEALGCKTVADLMKVDARTLIDTAAATVALRIFPVRDERILPLDPWEAYENGIAKDIVFLQGCNKDEMNCFVADWTVEGFKAWAADRKTKKYAHLLTDEERAKIESFCKEGAVEDWEPDSRLFGHSWFIEGAIKMSESQTKGGGKSYTYYYRVESSDPIRKSAHFEEVPIVFAHPEMIEGRQYDATFMKTMRKMWVQFAKTGNPSLSASESPDGKAKVWPLYDLKDKQVMVLDEFDIHPTKESDVKIVDWERTCFLTKYYML

SEQ ID NO: 12 - protein, 558 aa, unknown
  bacterial
MRNFKKWMLAAILICGTTMVLTSCSSKEDNPVPSNPEIDKDVMNKKITDGNYDKSLAVKCINGTFVGKKAENIISYKGIPFVGQQPVGNLRWKAPVDVTPSNDVYEAYNYGKTPVQSPGDASVNYGTSENCLYLNIWKADETATQKKPVIVWIYGGGFEVGGTTDPQYDCYNLVKENPDVIFVTLAYRVSFFGFLHLSHLPDGKDYQDAANLGLMDQLMALKWVHENIAGFGGDPDNVTIWGESAGGGSCSLLPLVKGSHKYFKRVIAQSGAPAQTRSTEQAIEITNKVMEELGCKTVADLMKVSAEEFTETWTRLYGFFQVLGIRTFPERDGKFLPLDPWEAYANGAAKDIEFLQGCNRDEMNTFLGVIGVDRWNEWASGRKAEKLAKHTDEEKALVESYCSEITGESYEPTVRLLSQYMFIAPQIRLSEEQAKGGGKSYTYYFRVESSAPLIKAGHITELSALFNHPEITFFTGRAYDETFCKTLRKMWIQFAKTGNPSLTADMSPDGKAKEWPLYDTGEKKVMVLDEFDIHPAKESELKIVDWERTYFLTNTYFN

SEQ ID NO: 13 - protein, 286 aa, unknown
  bacterial
MAIIVKSALHQMDTESYDKAMADKTDKRTTEIPAGIVFPREVAQYTIGTMQVFEVPAENEEKPVVLYLHGGAYVHNFTSQHWKAMAEWAKATGCGIVAPNYPLLPLHTAAEAHLLMMQLYRELLKGIPAHRILIMGDSAGGGFTLALAQQIRNDSLDLPRHLVLISPWVDVMGGDSSLQERDNWLTIDVLQKYGTAWAGQMDVADPMISPIYGDMEGLPPTDLYTGTWEVFYTDVCKTYDKMKAAGVDAHLHVAQKMGHVYPLWPSHEGGKARREIAEIIKTNSKN

SEQ ID NO: 14 - protein, 320 aa, unknown
  bacterial
MNMKLKKILQRVAAVIIAIIVAGGAVWLITGRSPMAIIVKSALHQMDTESYDKAMADKTDKRTTEIPAGIVFPREVSQYSIGTMQVFEVPAENEEKPVVLYLHGGAYVHNFTSQHWKAMAEWAKATGCGIVAPNYPLLPLHTAAEAHPLMMQLYRELLKGIPAHRILIMGDSAGGGFTLALAQQIRNDSLDLPRHLVLISPWVDVMGGDSSLQERDNWLTIDVLQKYGTAWAGQMDVADPMISPIYGDMEGLPPTDLYTGTWEVFYTDVCKTYDKMKAAGVDAHLHVAQKMGHVYPLWPSQEGRKARREIAEIIKTNSKN

SEQ ID NO: 15 - protein, 412 aa, unknown
  bacterial
MTERQHIVGFTFENMTPQAERFLRNKFHLNKVDSIDAKLEQELTTSMRMDLFYQTAECQLIPQAGGVRVVLSAGERKSLQFHAGVRYDTEEYAAILLGLDIPLKTAIPVETDITLRLGKRLMARGEITIHPRFFTRPTFSYTFHRNDVDVYTYGDLDYNIRYNQSQAEFLPINFDLRHFNLQMGLRWDFLHYRNKLGSESAKQVTLENQHFFSYRARLVLNTEDNWNFPTRGTRFNAEYAYLTNNFAQLDVIDAEGNKQGKTAGMSDVYANWRMSFTIGSRFTIQPMLYGRLLFGSVVPAVFGNTIGGEWFGHYVEQQMPFAGVGNLEYVHNQFVAAQLQAQQRIGKSSYVLLRVAAGQHAPKVKELLDYRTMIGAQGAFYYNTMFGPVGATLGYSNHTKKAYFFINLGYEF

SEQ ID NO: 16 - protein, 766 aa, unknown
  bacterial
MKKIVLMIVAVLMFGAANAKSEKEAIDNLPLAMDQAPNAKSQKSRATVGVVLSGGGAKGMAHIGVLKVLEKAGIPIDIVTGTSIGSIVGGLYAIGYNSHSLDSMVRKQDWTYVITDKEDLRKQSYLDRKKANTYFLTTGLTIGKHNLNAGGLIKGKNLAELFQNLFVGYTDSLDFNKDLKIPFACVATNIMDNSEIVFHSGRLPQAIRASMSIPAAFSPVRIGDMVLVDGGLKNNFPVDVAREMGAEVVIGVTLNGKPKTAEDITGTMKIVGQIIDVNCVNKYDENKALSDLWMNVDPHGYSTASFTSEAIDSLIRYGEEEAMRHWDEIIALKERIGIDSSFHPIIHHPVRPNAMTERQRVTSFSFENMTPQAERFLRQKFHLNKVDSIDAQMAQELTTSMRMDLFYQTAECQLVPQGDGVRVILSAGDRKSVQLHAGARYDTEESAALQVGLDIPLKTAIPVETDITLRLGKRLMARGEITVHPRSFTRPTFSYTFLRNDVDVYTRGDLDYNIRYNQLLAEFLPINFDLRNFNLQMGLRWDYFHYRNTLGSKSVSLGTIENEHFFSYRARMAFSSEDNWNFPTRGARFNAEYAYYTNNFAKLNVRDADGNKQGTTMGLSDVRANWRMSFSFNSRFTLQPMLYGRLLFGSVVATGFSNTIGGEWFGHYIEQQMPFAGIGNMEYVGRQFVSAQLQAQQRIGSNSYVLLRMAGGQQAAKVKEIFDRRTILGVQGAYYYNTMFGPVGATLGYSNYTKKGYFFINLGYEF

SEQ ID NO: 17 - protein, 491 aa, unknown
  bacterial
MDWTRYPYPCFYKVDVYSKTTGKVPNSPEYHRLKEEYTFENNYDVPTTAIPTYYKITAYGIFGKLSSDEIFVANPNYNNPIRPIPIYKYTADNPASPIPYLVWHSIPDGVLYELELLSGPPDEENTITLSTTNHLMSTQKVFTNGIQIDLRPYLNQPRLFWRVRALNLRKEPIGVFSTAEQIMVDSSKPIPNKPLLNNFDVMPGFKQPIYPVFHWIPMYGADRYEVELMAQPPVEENNTVATPHRAWAKTVTDSFSCYDEYPRRYAGKYYWRVRAVNLKGETIGVFSDTDTFEVENHLTRPFAAAFGDSITHGGGAVSFSPCSMQYSYTTYLDFPAINIGRSGDTSKMSLDRFDNDVLPIKPVNLMILTGSNCLRDSYITAEMVIADLEGIYQKCVSNDIRPIMLTLPPINPANIMLAFRTPTDPNWYSKMVKINAYIKTKPYYIDLEPYFYDSTHTFMDPAFANDGLHPDIMGKMIMAEVINSHKEVFKQ

SEQ ID NO: 18 - protein, 437 aa, unknown
  bacterial
MKFSNKIVVAVIVGLMSFSGVVVAEDINTEAIGNAIGNITNEIDTEKPLQTEPLNKVRISWETIPGAVSYRLVITDGDSASKDAEVKHFNLIPNNGYELDTANMFKAKDYYWRVCPLDYDGNVMGAYSDPKPLVGEEVNPKAPLATAEFEKMAYMPLYPVFSWIPVDGSGGYEIRVSRENKFSPGRYEVVRVMTTKNNVYYEEGGYTWPGHYMWQVRSQNKAGEPNTEWSEPKYFDVESHVRVAALGDSITHGGGVCNVPPGYAMYSWEYYCQVPVKNLGRSGDSVQDMAQRFDQDVMAFTPGMLIIMGGVNNFRAGEDGWDTIAAMEQILAECQYHNVIPVFVTATPINPSLMSKVPTIEPAAWNWKEQQQLLNQWIKNQPYHVDVTPALTDANGNLKAELTTDGLHPDHLGKKIIGEIISNYLLTTFPNYNLTAK

SEQ ID NO: 19 - protein, 247 aa, unknown
  bacterial
MIINGGEPFFLKGGPHGILLIHGFTGVPAEMLLLGRYLNDKGYTVLAVRLAGHGTTTEDMAHMTSEDWYDSVCDGYSILASICEKISVVGQSMGGLLSILLAANASVKCCCTLAAPIFIHEDKQLYRLPAKEYCKNKYWPKKKRMHFDVPQVCNASYGEYPMVSVHELLDLIDRTKRELPRVKCPMLVMHSHGDHTARDRSANKIYDLSGSYEKELVWLEESGHLITIGSEREKAFELVDEFIREHS

SEQ ID NO: 20 - DNA, 1749 bp, unknown
  bacterial
ttagttattatttttataagctgcaatgcgctttgctacgttttcctggaggtttttgtagaagaacgagtagtcgttttcgtggaagctggcaggtccgaagaactccttcccatcggtgacgggagctgtcgttttagtcacgatgacgccacgggcaaggtttatctgtgcatcgacgccgaggtcaacgaactcacgagcacctgtttctttgttctgcacgagcgaacccttgttctcactggcggatgcgtaggtttcgtcgagcttccaattcaacggattgatgcttatagcacccgggagcactgcgacgttcctggcgttctcctctacgttcttgggaccctcagtgttatagctgatgatgacacccgtgtcgctctctccggttgcaaacttcaggtgcggatatgcttcgaggtcatcttttgtgatggcaaagccaatggggtaggcagccaccatgcgctggtagtaatcgaggtgctctgagaagtagtttttcaatacatacctgaccatggccgagccttgactgtgaccagcaataatgaacggacgtccattgttacaattctcgaagtagtagtcgagcgatgctttgatgtcggtatatgataaaccagcaagcgctgcgtcgatgtttccggttttctttgcgatttcattggcatacctcatgccggcctggcggtagtatggtgcaaacacgttgcaagactcctcaaacaaggttgcattcgtctcatattcaccattggcgaacattatcatctcagggttgtcaatgggtacgtaattggaggcaccatcgtcgaagctcgactcaacatacgatgtcgagtagatgtagatggcatcaacgtctttggtaatttccggaaatgcaagccagttgaccttatcgctgtaatcgataatctccggctgtaccctcgtcatttggaatgaagcggtgtaagtcgagccgtcctcgcgcacacgcagcgccgtagccttcaccacatcatccttgatgctttcaatttcccaccactcacgcagttcctcactgtcttttgtgtcctgccagcgggtgcagagcagcgtgccatcgacaaagtagtctgccagatagtggggagcttccacccacttgtcgcccaccttgtcgtagaaagcataggtgccggcggccatgcattcccaacggtgcagctcatcgttggtttcgtcacccagatctctggtgaccttgccttcccacaagccatagaaggctgattcatagtcgtttttcacgcgttcccagcgctcctcttcataagcctcatggtgaacctctttgccatcaacaaggacactccagttggagctcagcaccatatccttatccgtaatggaggaaacggtaacatccagtacatgtgtggtatggtcatcttccttggctgtaatcaacattttattaccgtcgattttcacgtcggcttccacctcgtcgttccacgacttgctatagaaatcagaaagacttccgtatgctttcgtaggcgaatcgaatgtgacaacagttttcaagttggtcggacatgcttcgccattcagttctgcaaccatccattttccgacgattttctcagcaaggttgaggtctggctgaggaacgggggaggaattatcatcgttcgacgaacacgccgtgaatacacttatgccgcaaatcaggatggcggcaagcatccactttttcaattgtctcat

SEQ ID NO: 21 - DNA, 1743 bp, unknown
  bacterial
atgagacaattgaaaaagtggatgcttgccgccatcctgatttgcggcacaagtgtatttatggcgtgctcgtcgaacgaagacaaccccgccccccagccagacattaaccttgcagagaagattgtcggtaaatggatggtggccgaactgaatggcgaagcgtgtccaaccaacttgaaaactgttgtcacattcgattcgcctacgaaagcatacggtagtctttctgatttctatagcaagtcgtggaacgacgaggtggaagccgacgtcaagatcaacggcaataaaatactcatcactgcgaaggaagacgacaataccacacatgtgctggatgttaccgtttcctccattacagacacggatatggtgctgagttccgactggactgtccttgttgatggcaaagaggtttatcaggaggcctatgaagaggagcgctgggaacgcgtgaaaaacgactatgaatcagccttctatggcttgtgggaaggcaaggtcaccagagatctgggtgacgaaaccaacgatgagctgcaccgttgggaatgcatggccgccggcacttatgctttctacgacaaggtgggcgacaagtgggtggaagctccccactatctggcagactactttgtcgatggcacgctgctctgcacccgctggcaggacacaaaagacagcgaggaactgcgtgagtggtgggaaattgaaagcatcaaggatgatgtggtgaaggctacggcgctgcgtgtgcgcgaggacggctcgacttacaccgcttcattccagatgacgagggtacagccggagactatcgattacagcgataaggccaactggcttgcgtttccggaaattaccaaagacgttgatgccatctacatctactcgacatcgtatgttgagtcgagcttcgacgatggtgcctccaattacgtacccattgacaaccctgagatgataatgttcgccaatggtgaatatgagacgaatgcaaccttgtttgaggagtcttgcaacgtgtttgcaccatactaccgccaggccggcatgaggtatgccaatgaaatcgcaaagaaaaccggaaacatcgacacagcgcttgctggtttatcatataccgacatcaaagcatcgctcgactactacttcgagaattgtaacaatggacgtccgttcattattgctggtcacagtcaaggctcggccatggtcaggtatgtattgaaaaactacttctcagagcacctcgattactaccagcgcatggtggctgcctaccccattggctttgccatcacaaaagatgacctcgaagcatatccgcacctgaagtttgcaaccggagagagcgacacgggtgtcatcatcagctataacactgagggtcccaagaacgtagaggagaacgccaggaacgtcgcggtgctcccgggtgctataagcatcaatccgttgaattggaagctcgacgaaacctacgcatccgccagtgagaacaagggttcgctcgtgcagaacaaagaaacaggtgctcgtgagttcgttgacctcggcgtcgatgcacagataaaccttgcccgtggcgtcatcgtgactaacactacagctcctgtcaccgaagggaaagaattcttcgggccagccagtttccacgaaaacgactactcgttcttctataaaaacctccaggaaaacgtagcaaagcgcattgcagcttataaaaataataactaa

SEQ ID NO: 22 - DNA, 1059 bp, unknown
  bacterial
tcacttttccacgaacgccctaatgcgatcggcaatattctgcttcaggttgttgtagaagaaaccgtagtcgttgcggtggtaacactcaggcccgaacaagggtacagcctctttctcactgatggcaaagggcttggctgccgccgtggtgacgacgactgagccacggacggtgtcaacacgggcatcggcaatgccgggaacaatcaccttgcccgtcgtctcgtcgagcgagccgaggttgtccttgacgggggcgtaggtgtcgtcacgcttccaattgatggggttgatgctcaggccgtcgggagccagcgtagcgttcttggcatccttgttgccgacaccctcggtattccacgagacgatgacgcccgtgtcgatggctccctcggcaaacttcaaaccagtgcgctccaggaagtccttggtcacggcaaagccaatgggataggcagccaccatgcggctcagatgctcaggatgcttggtcatgtagttctccagcaggttgagcagcgtcgaggaaccctgcgagtgacccgccagaatgaatgggcgcccctgattcaggtgcgtcagaaagtagtcgagcgcatcggtggcatcgaaacccgacagatagttggtcacgaagcggtagtcagtgccaggcttaggcatcgaaatctggcgatagaagggcatgaagatgttgcagctctcttcgaacacgctggtctgaacctgtctgacaaacttggcaccagccaccatttcggagtcggtcacttcgcatatctggacagggtcgcggaaaccagttaccgtaggatagatgaagaacacatcgacaggcttcgttgcctcagtcggtctctccacccagttctcggccttactgtagtcgagcttggtctggaacttctctatgcctgcgacgtctttcagcacctcaggaatactgtcctgggcgtcggcgccggcggtttgtttgttactactcttgcacgaggcaacggtcatggctgtcagcatcgccgatgccagcagcacgaggaaattctgtcttttcat

SEQ ID NO: 23 - DNA, 1086 bp, unknown
  bacterial
tcacttttccacgaacgccctaatacgatcggcaatgttctgcttcaggttgttgtagaagaaaccgtagtcgttgcggtggtaacactcaggcccgaacaagggtacagcctctttctcactgatggcaaagggcttggctgccgccgtggtgacgacgactgagccacgaacggtgtcaacacgggcgtcaacaatgccaggcacgatgaccctgcccgtcatctcgtccagcgagcctaagttgtccttggcagaggcgtaggtgtcgtcacgcttccaattgatggggttgatgctcaggccgtcgggagccagcgtagcgttcttggcatccttgttgccgacaccctcggtattccacgagacgatgacgcccgtgtcggtggctccctcggcaaacttcaaaccagtgcgctccaggaagtccttggtcacggcaaagccaatgggataggcagccaccatgcggctcagatgctcaggatgcttggtcatgtagttctccagcaggttgagcagcgtcgaggaaccctgcgagtgacccgccagaatgaatgggcgcccctgattcaggtgcgtcagaaagtagtcgagcgcatcggtggcatcgaaacccgacagatagttggtcacgaagcggtagtcagtgccaggcttaggcatcgaaatctgacggtagtagggcatgtagatgttacaactctcttcgaacacgctagtctgcacccgacgtaccatctgggcaccggccaccatttccgagtcggtcacctcgcatatctgcacagggtcgcggaagcccgtcaccgtaggatagatgaagaacacgtcaaccggcttcttagcctcggctggtctctctacccagttttctgtcttgctatagtctagtttggtctgaaacttctctatgccggcaacgtccttcagcacctcaggaacactgtcctgagcagaagcgtcggtggcgggtttgttactgctcttgcacgaagcaaggctcatggctgccagcatcaccgatgccagaagcacaagggaattctgtcttttcatctttattattctatgagttgtttgcat

SEQ ID NO: 24 - DNA, 1059 bp, unknown
  bacterial
ttatttctccaggaatgccttgacgcggtcggcgacgttctgcttgaagttgttgaagaagaagccgtagtcgtggaggtggtaactctctgggccgaacaagggggcaccatcggcggggatggtgtatgtggcagcatcggccgttgtgacgatgacggagccacggacggtgtcaacacgggcatcggcaaagcctggggtgacgagctttccggttatgtcaagcgaaccaaggttgtcgctgacaggtgcgtaggtatcgtcgcgcttccagttaatcgggttgatgctgatgccaccaggggcaatcacgacgttcttggcatccttattggcagcaccctcggtgttccacgaaacgatgacgcctgtatcggtagcaccctcagcgaacttcagtcctgttcgcgctaggaaatccttggtcacagcgaaaccgacaggataggcagcaaccatacgcttcagagcctcaggatgcttcgtcatgtagttctccagcaggttaatcaacgtggaggctccctgcgagtggcctgcgaggatgaacggacggccctgattgaggttgttcaggaagtagtcgagggcatcggtagcatcgaagccagagaggtagttcgtgacagaagaataatctgtgccaggtttcggcatggagatctggcggtaataaggcatgaagacattgcagctctcgtcgaaaacactggtttgaatctgtctgaccatcttagcaccagccaccaactcggagtcggtgatttcgcaaatctgaaccggatcgcggaagccggtcacggtcggatagatgtagaacacgtcaacaggctttgtggcctccgcgggtttctccaaccaacactcgtccttgctgtagtcgagcttggtctggtatttctctaagccagcgacatccttcagaacctcgggaatgctatcctcggtcgcaacgtcggctgcgggtttgttactcatcttgcacgaagcaaggttcatggctaccagcatcgccgatgccaggagcacgaggatattctgtcttttcatgatcat

SEQ ID NO: 25 - DNA, 846 bp, unknown
  bacterial
atgatgagaagtctgaaggttctaatttgtgtcatctgcgtcatctgcggcttcatagctgccacacccgacaagaccactacaatcttcgtggttggcgattctaccgcagctaataaggacactaccggtggaaaggtagagcgtggttggggtatgatgctccagcaatgcttcgatgccgatttcatcgtggttgataaccacgctgtgaatggccgttcctcgaagagcttccttgatgagggtcgttgggataaggttttggaaaagattcagccgggtgactacgtactcattcagtttggtcataacgatgaaaaggcgcagcctgcccgtcataccgatcctggttccaccttcgactataatctcgcccgatatgtccgtgagacaagagaaatgggtggtatccctgtgctgatgaacgctgtggcacgtcgtaatttcttcattatgccgcctgaagtggctgacgatgaacagttgcgtaataccatctttagtgatagtgaacaccttgtcgagggtgattctctcaacgatacccacggcctctatcgtgtggctccccgtgatgtagcccgtcgtatgaactgccatttcatcgatgccaaccgcatcaccgaagaccttgagaaacgtcttggtcgtgagggctccaagaaactccatatgtggttccgtcccggtgaggaacccagcctgccccaaggcaagcaggacaatacacattataatatatatggtgcccaggtcgttgccaatctcctcgccgatgctctttgtgaggagattcctgtgctcaagccctatcgtgtgaagggagttcggaacgcaaaataa

SEQ ID NO: 26 - DNA, 1578 bp, unknown
  bacterial
tcaaggcatataatagttggtcaagaaataggtcctctcccaatccactatctttagctcagactccttagccgtatggatgtcgaactcgtcgagcaccatcacctgcctgtccttcatgtcgtaaagcggccactccttggccttgccgtcgggcgattcggctgcgctaagcgacggattgccggtcttggcaaactgaatccacatcttgcgcatggtcttgctgaaggtttcgtcgtatgctcttcccgtgaactcctgaatctccggatgggcaaatactactggcaactctgacgcatgtccggccttgatgtatggctgggaggattcaaccctgtagtagtaggtgtacgactttccaccggccttgaactgctcgtccgacagtctgatttgcggtgcgataaacatcatctgactgtacaggttggaggtagcctcatagttttctcccttgatgctgttcaggtagctctccaccaacgctttctcctggtctgtcagttgtgcgagcttctctgccgtgcgctctttggcccacgcattccaagcctcctcaccgagggctcccatgaaggtgttcatctcatccttgtcgcagccctgaaggaactcgatgtccttggcagcaccgttcgcgtaggcctcccatggatcaagcggcaggtagatgccgtctctctctggcatggtgcgtatgccgagtgcactatataacccgagaatgggagcccatgtcttcatgatatcctcggcgctgatcttcaacaggtcggcaacagtcttgcagccgagtttctccatcagactgttcgtgcatgcgatggcctgctccgcacttctcgtctgggcgggagcgccactttgtgcgatgacacgcttgaaatactgatgcgagcccttgatcagcggaagcagggtacagcttcctgcacctgccgactcaccccagatggtcacattgtcgggatcgccaccgaaagctgcaatattctcgtgaacccacttcaaggccatcagctgatccaaaaggcccaggttctgggcgtcaggatagtccttaccgtctgacaggtgagacaggtggaagaacccgaagacattgattctgtaattgagggtcacgacgatgacatcaggattttcattcaggaaattggtgagatcgtacatcgggtcggtcgctccgcctacatcgaaagcgccaccgtaaatccatacgataacaggtttcttagcagccgcctcatcagctttccagacgttcaggtacaggcagtcctcgcctgttccatattctgcagcggggtcgccaacctcttgcacgggagccttcccatagtaataggcctcatagacaccatcgtcgggcgttacatctaccggggctttccagcgcaggttgccgactggctgctgacctacaaacggaatgcctttataggaaatgatgttctccgcctttttgccgacgaaagttccattaatacatttgacagccaacgcttggtcgtatataccgtcaataatcttttgattctctacgtattgttccatataaaatctcatcat

SEQ ID NO: 27 - DNA, 1584 bp, unknown
  bacterial
ttaaagcatataatatttggtgaggaaataggttctctcccaatctacgatctttacctcagactccttagccggatggatgtcgaactcatcgagtaccatcacccgcttgttgtccagatcgtagagaggccattcttttgccttaccatcgggagagaggtccgcgctgagcgatgggttgccggtcttggcgaactgaacccacatcttgcgcatggtcttgcagaaggttgcatcgaattccctacccgtatcggcactcatctccggatgattgaatacgaccgaaagttctatggcatgtccgcatttcataatcggatcgggcgactcgggtgtgaagtaataggtgtatgccttgccaccgcccatcgtctggcactcagacaacttgatggtgggcacaataaaccagctctggctgaagagactgctgtctggctgaaagtaatcacccttcacatctttcatgtaactatcgaccagtgctttctcgtcagttggcaacttgtcatacttctcttgctttcgcgcagcaatccaattgtcccagccttcctttccgaagctcgacacgaagaagttcatctcgtccttgttgcaacctaccagaatctccaagtccttcgctgcaccgttggcgtatgcctcgaaggtgtcggtgggcagatactttccgtctctttcgggcatctggtgtgcaaagttgactgctgtagctgtttccagaagcttgtcgccatcgaccttcagaagatcggcaaccgtcttgcaaccgagcgcctccatcagtttgttggtgccttcgataccttcttccggagatctcgtctggttcacagaaccgctctcagcgatcaatcgcttgaaatacttctgtgagccaggaaccaacggctgcaaggtacagctggcagcacctgcggattcaccgaaaatggtcacattgtctggatcgccaccgaagccagcgatattctcatgaacccacttcagcgccatcttctgatccagaagtcccaagttctgtgagtcttggtaatccttaccgtcgggcaggtgagacaggtgcaggaagcccatgacgccgagtcggtaagcaatggtaacgactacgacttcgggattctccttcacgaaattgatgcagtcgaacatcgggtcaatggttccgcctgactcaaaagcaccgccgtgaatccataccataacgggtttcttatccgttgttgcgtcatcggccttaaatacattcagatacagactgtcttcatccaggtaatagagtgaaccaacctcaaacgacgggttgccgtagccgctcttcgcattgtaataagcctcatagacaccttcgttgggaacagcatccacaggcgccttccaacgcaggtcgcccacaggttgcttacccacataaggaatacccctatagacgatgatattatcattcttccgacctacgaaagtaccattgatacacttgacagccaacgacttgtcgtaattgccatcggtaatctgtttgttctcgccgccatacataccgtgcagcgcatctctaatctcttgagcatccat

SEQ ID NO: 28 - DNA, 1581 bp, unknown
  bacterial
atggatgctcaagagattagagatgcgctgcacggtatgtacggcggcgagaacaagccgattaccgacggtaactatgaccagtcgctggccgtgaagtgtattaacggtactttcgtaggtcggaagaatgataatatcatcgtctataggggtattccctatgtgggtaagcaacctgtgggcgacttgcgttggaaggctccagtggatgctgttcccaacgaaggtgtctatgaggcttattacaatgcgaagagcggctacggcaacccgtcgtttgaggtaggttcactctattacctgagtgaagacagtctgtatctgaatgtatttaaggccgatgacgcaacaacggataagaaacccgttatggtatggattcacggcggtgcttttgagtcaggcggaaccattgacccgatgttcgactgcatcaatttcgtgaaggagaatcccgaagtcgtagtcgttaccattgcttaccgactcggcgtcatgggctttctgcacctctctcatctacccgacggcaaggattaccaagactcacagaacttgggactcttggatcagctcaaggcgctgaagtgggttcacgagaatatcgcaggctttggcggcgaccccgacaacgtgaccatcttcggtgaatccgcaggtgccgccagctgtaccctgcagccactgcttccgggttctcatcagtatttcaagcgactgattgctgaaagcggttccgtgaaccagaccagatcaccggaagaagctatcgagtgtaccaacaaactgatggacgtgctcagctgcaagaccgttgccgatctcttgaaggtcagtggcgacaagctcctggaagcttctacagtaaactttgtacaccagatgccagaaagagacggaaagtatctgcccacagatacctttgcggcatacgccaatggtgcagcgaaggacttacagattttggtaggttgcaacaaggacgagatgaacttcttcgtgtcgagtttcggaaaggaaggttgggacaattggatcgctgcgcgtaagcaggagaagtttgaccagttgccagctgacgagaaagcactggtcgatagttacatgaaagatgtgaagggtgattactttcagccagacagcagtctcttcagccagagttggtttattgtgcccaccatcaagctgtctgagtgccagacgatgggcggtggcaaggcatacacctattacttcacacccgagtcgcccgatccgattatgaaatgcggacatgccatagaactttcggtcgtattcaatcatccggagatgagtgccgatacgggtagggaattcgatgcaaccttctgcaagaccgtgcgcaagatgtgggtacagttcgccaagaccggcaacccgtcgctcagcgcggacctctctcccgatggtaaggcaaaagaatggcctctctacgatttggacaacaagcgggtgatggtactcgatgagttcgacatccatcccgccaaggaatccgaagtaaagatcgtggattgggagaggacctacttcctcaccaaatattatatgctttaa

SEQ ID NO: 29 - DNA, 1563 bp, unknown
  bacterial
atggaacaatacgtaaagaatcaaaagataactgacggtcattacgacaagtccctggccgtcaagtgtattaacggaaccttcgtcggcaagaaggcggagaacgtcatttcctataagggcatcccgtttgtcggtcagcaacctgtcggcaacctgcgctggaaagccccggtagatatcgttcctgacgacagcatctatgaggcttattactatggcaaagctcccatccaggagccgggcgatcctgctgccgaatatggcaccagcgaggactgcctgtacctgaacgtctggaaagctgatgaggcggctgctaagaagcctgttatcgtatggatttacggtggcgctttcgatgtaggcggagcgaccgacccgatgtacaatctcaccaatttcctgaatgaaaatcctgatgtcattgttgtaaccattagttacagaatcaatgtcttcggctttttccacctgtcgcacctgtcagacggcaaggactatcctgacgctcagaacctgggccttttggatcagctgatggccttgaagtgggttcacgagaatattgcagctttcggtggcgatcccgacaatgtgaccatctggggtgagtcggcaggtgcaggaagctgtaccctgcttccgctgatcaagggctcgcatcagtatttcaagcgtgtcatcgcacaaagtggcgctcccgcccagacgagaagtgcggagcaggccatcgcatgcacgaacagtctgatggagaaactcggctgcaagactgtcgccgaccttttgaagatcagcgctgaggatatcatgaagacatgggcacccattctcgggttatatagtgcactcggcatacgcacaatgccagagagagacggtatctacctgccgcttgatccgtgggaggcctacgcgaacggtgctgccaaggacatagagttccttcagggctgcgacaaggatgagatgaacaccttcatgggagccctcggtgaggaggcttggaatgcgtgggccaaagagcgcacggcagagaagtttgcccagctgaccgataaggagaaggcattggtggagagctatctcaacgacatacagggagaaagctacgaacctaccgtcaacctgttcagtcagatgatgtttattgcaccgcaaatcagactatcggaggaacataccaagagtggaggtaagtcatataccttctactacagggtagaatcctcccagccttacatcaaggccggacatgcgtcagagctgccagtagtatttgcccatccggagattcaagagttcacgggaagagcatacgacgaaaccttcagcaagactatgcgcaagatgtgggtacagttcgccaagaccggtaatccgtcgcttagcgcagccgaatcgcccgacggtaaggccaaggagtggccgctttacgacatgaaggacaggcaggtgatggtgctcgacgagttcgacatccatacggctaaggagtctgagctaaagattgtggattgggagaggacctatttcctgaccaactattatatgccttga

SEQ ID NO: 30 - DNA, 1560 bp, unknown
  bacterial
tcaaagcatataatacttggtgaggaaacaggtcctctcccaatccacgatctttacatcggattcctttgtcgggtggatgtcgaactcgtcgagtaccatcacctgcttgtccttcaggtcgtagagtggccataccttggctttgccgtcgggagattcggacgcactaagtgacgggttgccggttttggcaaactgcacccacatcttgcgcatggtcttcatgaaggtggcatcatactggcgaccctcgatcatttccggatgagcaaacacgatgggaacctcttcgaaatgtgcactcttccggatagggtctgaagactctactctataataataggtataggacttgccaccgcccttggtctgactctccgacattttgatggcgccctcgataaaccagctatggccgaacaagcggctgtcgggctcccagtcttctaccgctccttctttacagaacgactcaatcttcgctctctcctcatccgtgagcagatgggcatacttcttcgtcttgcggtcagcagcccacgctttgaatccttccaccgtccagtcagccacgaaacagttcatctcgtccttgttgcagccctgtaggaacacgatgtcctttgcaatgccgttttcgtaagcctcccacggatccaaaggcaggatacgttcgtctctaacggggaagatgcgtaatgcgacggtagcggcggctgtatcgatcagcgtgcgggcatcgactttcatcaggtcggcaacggtcttgcaaccgagggcctccatcaaatcattcgtggtggagatgctctcttccacagaattcatctgtgagggggcgccgctttgtgcgatgaccttcttgaaatactgctgggaacctttgatcagcggctgcatggtcacgcttccagcaccggcagattcaccccagatggtcacgttgtcgggatcgcctccgaagcctgctatgttctcatgaacccacttcaatgccattttctggtccaacagtccaaggttctgtgcgtctggatagtccttgccatcagccaaatgtgacaaatggaagaagccaagaacaccgagtctgtatgcaactgtaacaacaatgacatcaggattccctttgacaagttcggtgcaatcaaacagctctatgcctgttccgcctgccacaaaagcaccaccgtgaatccataccatgactggtttcttcacctctgatgcatcatcagccttataaacattcaaatacaggcagtcttcaccctgatggtcagtgattgacaggtcctgaacggacattttctcaaaatgataggcttcatataccccatcgttcgaagagtactccactggggctttccagcgcagttccccgataggttgctgaccgacaaagggaatgcctttgtaagaaatgatattctccgtctttttgccgacgaaaataccgttaatacacttgacggccaacgatttgtcgtaattaccgcctgtaatcttttggttctcactatactgagctctcagttgctcttccttttcactcat

SEQ ID NO: 31 - DNA, 1677 bp, unknown
  bacterial
tcaattgaaataagtgttggttaggaagtaggtacgctcccagtcaacaatcttcagctcggactccttggccggatgaatgtcgaactcatctaagaccattactttcttttctcctgtgtcgtagagtggccactctttggccttgccgtcaggagacatgtctgctgtgagcgaaggattgccagtcttagcgaactgtatccacatcttgcgcagggtcttacagaaggtttcgtcgtatgctcttcctgtgaagaacgttatctccggatggttgaacaatgccgacaattctgttatatgtccggcttttatcaatggcgcggaagattctaccctaaagtaataggtgtatgacttgccgccacccttggcttgctcctctgagagtctgatctgtggtgcgatgaacatgtactgactgaggagacgaacggtaggttcgtaactctctcctgtgatttcgctgcagtagctctcaaccagtgccttctcctcgtccgtatgcttggcgagcttctctgccttgcgcccactggcccactcattccaacggtccactccgatgacacctaagaaagtgttcatttcatccctattgcagccctgaaggaactctatgtctttagctgctccgtttgcgtatgcctcccatggatcaaggggcaggaatttcccgtctctctctgggaatgtgcgtataccgagtacttggaagaaaccatacaagcgtgtccacgtttctgtaaactcctcggcgctaaccttcatcaggtcagcgacggtcttgcagccgagttcctccattactttattcgtgatttcgatggcctgctctgttgacctcgtctgagcgggggcaccactctgcgcgatgacacgcttaaaatacttgtgggagcctttgaccagaggaagcagactacaacttccaccaccggcagattcaccccagatggtcacgttgtcggggtctccaccaaagccggcaatgttctcatgcacccacttcaatgccatcagctggtccatgagtcctaagttcgctgcgtcctggtagtctttgccatcgggcaggtgcgaaagatgcaggaaaccgaagaagctgaccctgtaagcgagggtcacgaaaatgacatcaggattctctttcacgaggttgtagcaatcgtactgagggtcagttgttccgcctacctcaaagccgccaccgtaaatccacacaatgaccggcttcttctgcgtagccgtttcgtcagccttccaaatgtttaggtacaggcagttctcgctcgtgccataattaacagaggcgtcgccgggcgattggactggagtcttcccataattataggcctcatacacgtcgttgctcggagtaacatctactggtgccttccaacgcaggttgccgacaggctgctggccgacgaacgggatgcccttgtaggaaataatgttctccgccttcttgccgacgaaggtgccattaatacatttgacagccagcgacttgtcataattgccatctgtaatctttttgttcattacgtccttgtcaatctcaggatttgaaggaacagggttgtcttctttgctactgcatgaagtgagcaccattgttgtgccgcaaattaggatggcggcaagcatccactttttaaaatttctcat

SEQ ID NO: 32 - DNA, 930 bp, unknown
  bacterial
ttaatttttagagttcgttttaataatttctgctatctcacgacgggctttgccaccctcgtgactgggccagaggggatagacatgccccatcttttgggcgacatgcaggtgagcatcaacaccagcggccttcattttgtcgtaggtcttgcagacatcggtgtagaacacctcccatgtaccggtatagaggtcggtaggcggtaaaccttccatgtcaccatagattggcgaaatcatagggtcggcaacatccatctgtcctgcccaagcagtgccatacttctgcaacacatcaatggtgagccagttgtcgcgctcctgcagcgaagaatcaccgcccatgacatcaacccagggcgagataagcaccagatggcgaggcaggtcgagcgagtcattgcgtatctgctgtgccagcgccagcgtaaagccgccacctgcagagtcgcccatgataagaatacggtgggcaggaatacccttcagcagttcacgataaagctgcatcatgagcaggtgagcctcagcagcagtatggagaggcagcaacgggtagttgggagccacgatgccacagcctgtggcctttgcccactcagccatggccttccagtgctgagacgtaaagttatggacgtaagcgccgccatgcagatagagtactacaggtttctcttcgttctcagcaggcacttcaaatacctgcatggttccgatggtatattgagcaacctcacgcggaaatactatgccggcaggaatctccgttgtacgcttgtcggtcttgtcagccatggccttgtcgtacgactccgtgtccatctggtgtaaagcgctcttcactatgatggccatgggtgagcgtccggtaatcaaccaaactgcgccaccggcgacgatgatggcgatgattaccgcagccac

SEQ ID NO: 33 - DNA, 963 bp, unknown
  bacterial
ttaatttttagagttcgttttaataatttctgctatctcacgacgggctttgcgaccctcttgactgggccagaggggatagacatgccccatcttttgggcgacatgcaggtgagcatcaacaccagcggccttcattttgtcgtaggtcttgcagacatcggtgtagaacacctcccatgtaccggtatagaggtcggtaggcggtaaaccttccatgtcaccatagattggcgaaatcatagggtcggcaacatccatctgtcctgcccaagcagtgccatacttctgcaacacatcaatggtgagccagttgtcgcgctcctgcagcgaagaatcaccgcccatgacatcaacccagggcgagataagcaccagatggcgaggcaggtcgagcgagtcattgcgtatctgctgtgccagcgccagcgtaaagccgccacctgcagagtcgcccatgataagaatacggtgggcaggaatacccttcagcagttcacgataaagctgcatcatgagcgggtgagcctcagcagcagtatggagaggcagcaacgggtagttgggagccacgatgccacagcctgtggcctttgcccactcagccatggccttccagtgctgagacgtaaagttatggacgtaagcgccgccatgcagatagagtactacaggtttctcttcgttctcagcaggcacttcaaatacctgcatggttccgatggaatattgagaaacctcacgcggaaatactatgccggcaggaatctccgttgtacgcttgtcggtcttgtcagccatggccttgtcgtacgactccgtgtccatctggtgtaaagcgctcttcactatgatggccatgggtgagcgtccggtaatcaaccaaactgcgccaccggcgacgatgatggcgatgattaccgcagccactctttgcagtatttttttcaatttcatgttcat

SEQ ID NO: 34 - DNA, 1239 bp, unknown
  bacterial
ttaaaattcatatccgaggttgatgaagaaataagctttcttggtgtggttgctataacctaaggtagcaccgacagggccaaacattgtattataatagaaggcaccctgggcacctatcattgtacgatagtctaagagttctttcacttttggagcatgttgaccggcagccacgcgcagcagcacgtagctgcttttgccgatacgctgctgagcctgaagctgagcggctacgaactgattgtgtacgtactccagattgccgacgccagcgaatggcatctgctgctcaacgtagtggccgaaccactcgccaccgatggtgttgccgaatacggcgggaacgacggagccaaagagcagacgaccatagagcataggctggatggtgaatcggctgccgatagtaaacgacatacgccagttggcatagacatcgctcatgcctgccgtcttgccctgtttgttgccctcagcatcaatgacgtcgagctgggcgaagttattggtcagataagcatattcagcgttaaagcgggttccgcgtgtggggaagttccaattatcctcagtgttaaggaccaaacgggcacgataactgaagaagtgctggttttcgagtgtgacctgtttagccgactcagatccgagcttgttgcgatagtgcaggaagtcccagcgcagtcccatctgcaggttgaaatgacgcaggtcgaaattgatgggcaggaattctgcctgtgactggttgtatcggatgttatagtccagatcgccgtatgtatatacgtcaacatcgtttctgtggaacgtatagctgaaagtggggcgtgtaaagaatcgggggtggatggtaatctcgccacgagccatcagtcgcttacccagtctgagcgtgatgtcggtttcgacagggatggccgtcttcagcggtatgtcaagtcccaacaggatagcggcatattcttccgtatcatagcgcacgccggcatgaaactgcaaagacttccgttcacctgccgagaggacgacacgcacaccgccagcttgaggtatcagttggcactcagctgtctggtagaagagatccatacgcatcgacgttgttagttcctgttccagttttgcgtcgatactatcaaccttgttcagatggaacttattcctcaagaaccgctctgcctgtggcgtcatgttttcgaaagtaaaaccgacgatatgctggcgctcagtcat

SEQ ID NO: 35 - DNA, 2301 bp, unknown
  bacterial
ttagaattcatatccgaggttgatgaagaaataacctttcttggtgtagttgctatagcccaaggtagcacccacggggccaaacattgtattgtaatagtaagcgccctggacaccgagtatggtgcgacggtcaaagatttccttcaccttggctgcctgctggccgcctgccatacgcagcagcacgtaactattgctgccaatgcgctgctgggcctgaagctgggccgagacgaactgacggcctacgtactccatattgccgatgccggcgaacggcatctgctgctcgatgtagtgaccgaaccactcgccgccgatggtgttgctgaaaccggtggccacgaccgaaccaaagagcaggcggccatagagcatgggctggagggtgaagcggctgttgaaggagaacgacatgcgccagttggcacgcacatcgctcaatcccattgtcgtgccctgcttgttgccgtcggcgtcgcggacgttgagcttggcgaagttgttggtataataggcgtattcagcattgaagcgggcgccacgtgtggggaagttccagttgtcctcgctgctgaaggccatacgggcacggtagctgaagaaatgctcgttctcgattgttcccagactgaccgacttagagccgagcgtgttgcggtagtggaagtagtcccagcgcagacccatctgcaggttgaaattgcgcaggtcgaaattgatgggcagaaattctgccagcagttggttgtaacggatgttgtagtcgaggtcgccccgagtatagacgtcgacatcatttctgaggaacgtatagctgaacgtcgggcgggtgaaggaccgggggtggacggtaatctcgccacgggccattaggcgcttacccagtcggagcgtaatgtcggtctcgacagggatggccgtcttcagcggaatgtcgagtcccacctgcagagcggcagattcctccgtatcgtagcgcgcaccggcatgaagctgcaccgacttgcggtcacctgccgaaaggatgactcgcacgccatcgccctggggcaccagctggcactcagccgtctgatagaaaaggtccatacgcatcgacgtcgtcagttcctgcgccatttgggcatcgatgctgtcaaccttgttcaggtggaacttctggcgcaggaagcgctctgcctgcggggtcatgttctcgaaagagaagctggtgacgcgctgacgctccgtcatggcgttcgggcgcaccgggtgatggatgatggggtggaacgaactgtcaatgccgatgcgctccttcagggcgataatctcgtcccagtggcgcatggcttcctcctcgccatagcgtatcagcgagtcgatggcctcagacgtaaagcttgcggtagagtaaccgtgagggtcaacgttcatccacaggtcgctgagggccttgttctcgtcgtacttgtttacgcagttcacatcaataatctggcctacgattttcatggtgcccgtaatgtcctcagccgtcttcggcttgccgttcagcgtgacgccgatgacaacttcagcacccatctcgcgggccacgtcaacggggaagttattcttcaggccgccatctaccagcaccatgtcgccgattctgacaggcgagaaggcggcagggattgacatcgaggcacgtatggcctgcggcaaccggccgctatggaacactatctcgctgttgtccataatgttggtagccacacaggcaaagggaatcttcaggtctttattgaagtcgagcgagtcggtgtagcccacaaagagattctggaacagctcagccaggttcttgcccttgatgagaccgccggcattcaggttatgcttgccgatggtcaggcccgtggtcaagaagtaagtgttcgccttcttgcggtccaggtaactctgtttgcgcaggtcttccttgtcagtaatcacataggtccagtcctgtttccttaccatcgagtcaagcgaatgagagttgtagccgatggcatagagaccaccaacgatgctgcctatcgatgtgccggtaacgatgtcgatgggaatgcccgccttctccagcaccttcagcacaccgatatgagccataccctttgcaccgccgccactaagcacgacgccaactgtggccctcgacttctggctcttagcatttggcgcttggtccatagctaacggcaaattgtcaatagcctctttctctgacttagcatttgctgcaccaaacatgagtacagcaacaatcatcaatactatctttttcat

SEQ ID NO: 36 - DNA, 1476 bp, unknown
  bacterial
ttattgcttaaatacttccttgtgggaattaataacctccgccataatcattttgcccattatatcaggatgaagcccgtcattggcaaaggccggatccataaaggtgtgagtggaatcatagaaatatggttccagatcaatataatatggcttggtcttaatgtaggcattgattttcaccattttggaataccagttaggatcggtaggtgttctgaatgccagcattatgttagccggattaattggcggcagtgtaagcataataggcctgatatcgttggatacacacttttggtaaattccctccaggtctgcaattaccatttcagcggtgatataggaatccctaagacaatttgagcccgtaagtatcataagattaactggtttaattggcaaaacatcattgtcaaagcgatcaagggacatttttgatgtatcgccactgcgaccaatattaatggcagggaaatcaagatatgtggtatagctgtattgcatactacaaggagaaaaggatacagcaccgccgccgtgagtaatactgtcgccaaaggctgcagcaaacggacgggtgaggtgattttcaacctcgaaagtatctgtatcggagaatacaccaatggtttcgccctttaaatttacagcccgtacacgccaataatattttccggcatatcttctaggatattcatcataacagctgaaggaatcggttactgtctttgcccaggcacgatgaggtgtggccactgtgttgttttcctccacaggaggctgtgccataagctctacctcatatctgtcagcaccatacattggtatccagtggaatacaggataaattggctgcttaaagcctggcattacatcaaaattattaaggagaggcttattaggtattggcttggatgaatccaccataatctgctcagctgtggagaatacgccaataggctcctttcggagatttagtgccctgacacgccagaaaaggcgaggctgattcagataaggtctgagatctatttgaataccattggtaaagaccttctgggttgacatcagatggttggtcgtggataaggtaatagtattttcctcatccggtgggccagaaagaagctccagctcataaagaactccatcgggaatgctgtgccatacaagatatggaatagggctggccggattatctgcagtatatttatatattggaataggtctaattggattattgtagtttggatttgctacaaagatttcatcagaggacagcttgccaaaaataccataggctgtaatcttatagtaggtaggtatcgcagtagtaggaacatcatagttattttcaaaggtatattcttcttttaaccgatgatattcagggctgttcggcaccttaccagtggtttttgaataaacatccaccttataaaagcaaggatatggataacgggtccaatccat

SEQ ID NO: 37 - DNA, 1314 bp, unknown
  bacterial
ttattttgctgttaaattgtaatttggaaaggtggtcagcagatagttgctgatgatctcccctattatttttttgcccagatggtccggatgaaggccatcagtagtgagctctgctttcagatttccgttggcatcagtgagggccggagtcacatccacatgatatggctgattctttatccactgattcagaagctgctgctgctccttccagttccaggccgccggttcaatggtaggtacctttgacattaaactaggatttataggagtcgcggttacaaaaacgggaatcacattgtggtactggcattctgccaatatctgctccatggcagcaatggtatcccagccgtcctcgccggctcggaagttatttacgccccccattattatcagcatccccggagtaaaagccatcacatcctggtcaaagcgctgggccatatcctgaacactgtcaccacttcgtcctaaattcttaacaggaacctggcaataatattcccagctgtacatagcataaccaggaggaacattacatacgccaccaccatgggtaatactgtcacccagagctgctaccctcacatggctctccacatcaaaatattttggttcagaccattcagtatttggctctcccgccttgttctgtgaacggacctgccacatataatgacctggccaggtgtagccaccttcttcatagtaaacattattcttggtggtcattacccgaaccacttcataccgtccaggagaaaatttattttcacggctgaccctgatttcatagccaccgctgccatcaacaggaatccaggaaaacaccggatacagaggcatatatgccattttctcaaactccgcagtagccaacggagcttttggatttacctcctcaccaacaaggggcttagggtcgctgtaagctcccatgacattgccgtcataatccaaaggacacacccgccaataataatccttggctttaaacatattggcagtatccaactcataaccattatttggtatgagattaaaatgctttacctccgcatctttgctggcggagtccccatctgttataaccagacgatagcttactgcccccggaatggtttcccagctaattctgaccttattcagtggctcagtctgaagtggtttttcagtatcaatctcgttagttatatttccaatggcgttaccaatagcctcagtattaatatcctctgccacgaccaccccggaaaaggacattaggccaactataaccgccaccacaattttattggaaaacttcat

SEQ ID NO: 38 - DNA, 744 bp, unknown
  bacterial
atgattattaacggcggcgagcccttcttcctaaagggagggccacacggcatacttttaattcacggctttaccggagttccggcagaaatgcttttgttgggcagatatcttaacgacaagggttacacagtgctggcagtgcgtctggccggacatggcacaaccactgaggatatggctcacatgacctcggaggattggtatgattctgtgtgcgatggttattccatattagcttcaatatgtgaaaagatttccgtggtgggacagtcaatgggaggcttgctcagcattcttcttgccgccaatgccagcgtgaagtgctgctgtaccttggctgcgccgatatttattcacgaggataagcagctgtatcgtttgccggccaaagaatattgcaaaaataaatactggcctaaaaagaagcggatgcatttcgatgtgcctcaggtgtgtaatgcttcctacggtgagtatccaatggtgtccgtacatgagcttctggaccttattgacagaaccaagcgggagcttccgcgggtaaaatgcccgatgctggtgatgcacagccacggcgaccacaccgcccgtgaccgcagtgccaacaaaatttatgatttgtctggaagctatgaaaaggaattggtgtggctggaagagtccgggcatttgataaccatagggtctgaaagggaaaaggccttcgagctggtggatgaatttataagggaacattcataa

SEQ ID NO: 39 - DNA, 27 bp, unknown
  bacterial
atgagacaattgaaaaagtggatgctt

SEQ ID NO: 40 - DNA, 24 bp, unknown
  bacterial
agcatccatagttttgtttccttt

SEQ ID NO: 41 - DNA, 21 bp, unknown
  bacterial
tcgaacgatgataattcctcc

SEQ ID NO: 42 - DNA, 24 bp, unknown
  bacterial
atgagacaattgaaaaagtggatg

SEQ ID NO: 43 - DNA, 21 bp, unknown
  bacterial
gcgtgctcgtcgaacgaagac

SEQ ID NO: 44 - DNA, 24 bp, unknown
  bacterial
ctcttgagcatccatagttttgtt

SEQ ID NO: 45 - DNA, 27 bp, unknown
  bacterial
aatatgaaaagacagaatttcctcgtg

SEQ ID NO: 46 - DNA, 21 bp, unknown
  bacterial
tcgtgcaagagtagtaacaaa

SEQ ID NO: 47 - DNA, 21 bp, unknown
  bacterial
ttctcacttttccacgaacgc

SEQ ID NO: 48 - DNA, 24 bp, unknown
  bacterial
tccggttgatgaagaaatatgcaa

SEQ ID NO: 49 - DNA, 24 bp, unknown
  bacterial
tcgtgcaagagcagtaacaaaccc

SEQ ID NO: 50 - DNA, 21 bp, unknown
  bacterial
ttctcacttttccacgaacgc

SEQ ID NO: 51 - DNA, 21 bp, unknown
  bacterial
gccaggctactggacaagaat

SEQ ID NO: 52 - DNA, 21 bp, unknown
  bacterial
tcgtgcaagatgagtaacaaa

SEQ ID NO: 53 - DNA, 21 bp, unknown
  bacterial
ttgttatttctccaggaatgc

SEQ ID NO: 54 - DNA, 21 bp, unknown
  bacterial
atgatgagaagtctgaaggtt

SEQ ID NO: 55 - DNA, 21 bp, unknown
  bacterial
gccacacccgacaagaccact

SEQ ID NO: 56 - DNA, 21 bp, unknown
  bacterial
aacttattttgcgttccgaac

SEQ ID NO: 57 - DNA, 24 bp, unknown
  bacterial
gattgggagagaacctatttcctc

SEQ ID NO: 58 - DNA, 24 bp, unknown
  bacterial
ccgtcaaggcatataatagttggt

SEQ ID NO: 59 - DNA, 24 bp, unknown
  bacterial
actatggatgctcaagagattaga

SEQ ID NO: 60 - DNA, 27 bp, unknown
  bacterial
ttaaagcatataatatttggtgaggaa

SEQ ID NO: 61 - DNA, 24 bp, unknown
  bacterial
actatggatgctcaagagattaga

SEQ ID NO: 62 - DNA, 24 bp, unknown
  bacterial
cgtattgttccatattcgttttca

SEQ ID NO: 63 - DNA, 21 bp, unknown
  bacterial
ggagaggacctacttcctcac

SEQ ID NO: 64 - DNA, 21 bp, unknown
  bacterial
agcgagcccaattggtgtgat

SEQ ID NO: 65 - DNA, 21 bp, unknown
  bacterial
ggctgttcctgttacacataa

SEQ ID NO: 66 - DNA, 21 bp, unknown
  bacterial
ggtaacttgaacctgtcaaag

SEQ ID NO: 67 - DNA, 30 bp, unknown
  bacterial
atgagaaattttaaaaagtggatgcttgcc

SEQ ID NO: 68 - DNA, 24 bp, unknown
  bacterial
acttcatgcagtagcaaagaagac

SEQ ID NO: 69 - DNA, 21 bp, unknown
  bacterial
agtgatcgcggaaaacagtca

SEQ ID NO: 70 - DNA, 21 bp, unknown
  bacterial
agagtggctgcggtaatcatc

SEQ ID NO: 71 - DNA, 21 bp, unknown
  bacterial
gcccgacgtaagtattcctat

SEQ ID NO: 72 - DNA, 21 bp, unknown
  bacterial
gcgttcgtggaaaagtgagaa

SEQ ID NO: 73 - DNA, 21 bp, unknown
  bacterial
gcccgacgtaagtattcctat

SEQ ID NO: 74 - DNA, 21 bp, unknown
  bacterial
ccactgcgaccgaatgccatg

SEQ ID NO: 75 - DNA, 21 bp, unknown
  bacterial
cctccgttgaatctgtcgatt

SEQ ID NO: 76 - DNA, 24 bp, unknown
  bacterial
gacgcccctggaatatagaattaa

SEQ ID NO: 77 - DNA, 21 bp, unknown
  bacterial
gctaagtcagagaaagaggct

SEQ ID NO: 78 - DNA, 24 bp, unknown
  bacterial
ttagaattcatatccgaggttgat

SEQ ID NO: 79 - DNA, 21 bp, unknown
  bacterial
accatggattggacccgttat

SEQ ID NO: 80 - DNA, 27 bp, unknown
  bacterial
tgcttattgcttaaatacttccttgtg

SEQ ID NO: 81 - DNA, 24 bp, unknown
  bacterial
gggagagataacatgaagttttcc

SEQ ID NO: 82 - DNA, 21 bp, unknown
  bacterial
gcagaggatattaatactgag

SEQ ID NO: 83 - DNA, 21 bp, unknown
  bacterial
ttcgtgagacagcttttttta

SEQ ID NO: 84 - DNA, 21 bp, unknown
  bacterial
accagcatgattattaacggc

SEQ ID NO: 85 - DNA, 21 bp, unknown
  bacterial
caccggctctctatgtgttat

(19) United States
(12) Patent Application Publication
(10) Pub. No.: US 2016/0060608 A1
(43) Pub. Date: Mar. 3, 2016

(54) LIPASES, POLYNUCLEOTIDES ENCODING THEM AND THEIR USES

(71) Applicant: ABERYSTWYTH UNIVERSITY, Aberystwyth, Ceredigion (GB)

(72) Inventors: Florence PRIVE, Jarrie (FR); Charles Jamie NEWBOLD, Aberystwyth (GB); Sharon HUWS, Machynlleth (GB); Nigel SCOLLEN, Ceredigion (GB)

(21) Appl. No.: 14/428,439

(22) PCT Filed: Sep. 16, 2013
(86) PCT No.: PCT/GB2013/052414

§ 371 Date: Mar. 16, 2015

(30) Foreign Application Priority Data
Sep. 14, 2012 (GB) 1216482.8

(51) Int. Cl. C12N 9/20; A61K 39/395; A61K 38/46; C12P 7/64; C07K 16/40

(52) U.S. Cl. CPC C12N 9/20; C12P 7/64; C07K 16/40; A61K 38/465; A61K 39/3955; C12Y 301/01003; C07K 2317/21; C07K 2317/76

(57) ABSTRACT

A lipase comprising a polypeptide or peptide comprising an amino acid sequence selected from the group consisting of SEQ ID NO: 1-19 and the polynucleotides encoding the lipases is disclosed. The lipases and nucleic acid sequences encoding these lipases can be used in the preparation of polymers, in formulations which may comprise detergents, as catalysts, in the preparation of bioplastics, as part of diagnostic kits, in the preparation of biofuels and for the prevention and/or treatment of a disease.

### TECHNICAL FIELD

The present invention relates to novel lipases isolated from the bovine rumen metagenome and from Anaerovibrio lipolytica 5S and polynucleotides encoding the lipases. The invention also relates to nucleic acid constructs, vectors, and host cells comprising the lipases as well as methods of producing and using the lipases.

### BACKGROUND OF THE INVENTION

The rumen is the primary site of lipid hydrolysis and transformation in ruminants, and lipid metabolism plays a significant role in regulating the overall lipid composition of microbial cells and also of milk and meat of the ruminant animal (Harfoot and Hazlewood, 1997; Scollan et al., 2006; Lourengo et al., 2010). The lipid content of forage ingested by ruminants ranges from 2 to 10% of the total dry weight (Harfoot and Hazlewood, 1997), which represent 1.5 kg of ingested lipids through forage per day by dairy cattle (Harfoot, 1978) and nearly 550 kg of lipid per year (Jarvis and Moore, 2010).

Dietary lipids enter the rumen either as triglycerides (neutral lipids) in concentrate-based feeds or as glycolipids or phospholipids (polar lipids) in forages (Harfoot and Hazlewood, 1997; Bauman et al., 2003). Other polar lipids, like sulfolipids, are also present as minor components in forage (<5%) (Harfoot and Hazlewood, 1997). On entering the rumen, lipids are hydrolyzed by lipases, which results in the liberation of glycerol and unsaturated and saturated fatty acids. These fatty acids go through microbial biohydrogenation and are transformed to more saturated end products.

Research on lipid metabolism in the rumen has largely focused on biohydrogenation of polyunsaturated fatty acids with an emphasis on the intermediates formed, in particular conjugated linoleic acids, due to the potential of these molecules to affect human health (Enjalbert and Troegeler-Meynadier, 2009). There is a dearth of data on microbial lipolysis, the first step in lipid metabolism in the rumen. It is known that dietary lipids are predominantly hydrolyzed in the rumen by obligatory anaerobic bacteria (Jenkins et al., 2008) and there is no evidence of rumen protozoa or fungi being significantly involved in ruminal lipolysis (Harfoot and Hazlewood, 1997; Jenkins et al., 2008, Lourengo et al., 2010).

However, to date, only six pure cultures of obligatory anaerobic, lipolytic bacteria have been isolated from the rumen, belonging to the genera Anaerovibrio, Butyrivibrio, Clostridium and Propionibacterium (Jarvis and Moore, 2010), and only a small number of lipolytic enzymes have been retrieved from the rumen metagenome of either cattle (Liu et al., 2009) or sheep (Bayer et al., 2010).

Hobson and Mann (1961) isolated a bacterium from the sheep rumen able to hydrolyze linseed oil triglycerides to glycerol and fatty acids, using anaerobic techniques and a combination of differential and selective media (Stewart et al., 1997). It was named Anaerovibrio lipolytica (Hungate, 1966), or A. lipolyticus (Strompl et al., 1999). The growth characteristics of strain 5S were described in continuous culture. Ribose, fructose and D-lactate were used as growth substrates and glycerol was fermented to propionate, lactate and succinate (Hobson and Summers, 1966, 1967; Henderson et al., 1969). Extracellular lipase activity was characterized in cell free-medium and after purification by chromatography on Sephadex columns; the lipases were most active at pH 7.4 and 20 to 22° C., and diglycerides were hydrolyzed more rapidly than triglycerides (Henderson, 1970, 1971; Henderson and Hodgkiss, 1973). Ruminal lipase activity in animals receiving mainly concentrate feeds is thought to be accomplished mainly by A. lipolytica, although other lipolytic species might be expected to predominate in grazing animals as A. lipolytica lacks the ability to hydrolyze galacto- and phospholipids (Henderson, 1971). These latter lipids are known to be hydrolyzed in vitro by the Butyrivibrio fibrisolvens strains S2 and LM8/1B (Harfoot and Hazlewood, 1997). A. lipolytica was shown to be present at around 10⁷/ml in grazing animals (Prins et al., 1975). Thus it has been hypothesized that A. lipolytica could have other activities, such as the fermentation of glycerol (Rattray and Craig, 2007). Other molecular microbial ecology studies have enumerated A. lipolytica in the rumen under different dietary conditions (Tajima et al., 2001; Koike et al., 2007; Huws et al., 2010) with similar conclusions. These studies have shown that A. lipolytica is predominant and one of the best recognized ruminal lipolytic bacteria. However no recent studies have been undertaken to enhance our knowledge of its lipase/s or genomic features using available modern laboratory techniques.

As lipases are used in a wide variety of applications, such as processing of fats and oils, detergent compositions and diagnostic reagents, there is a need for new and alternative lipolytic enzymes and their uses in biotechnological applications.

### STATEMENT OF THE INVENTION

It has been found that the bovine rumen metagenome harbours a number of active lipases. As such, these lipases and nucleic acid sequences encoding these lipases can be used in the preparation of polymers, in formulations which may comprise detergents, as catalysts, as part of diagnostic kits, in the preparation of biofuels and for the prevention and/or treatment of a disease.

In one aspect, the present invention relates to a lipase comprising a polypeptide or peptide comprising an amino acid sequence selected from the group consisting of SEQ ID NO: 1-19, or an amino acid sequence that has at least about 45%, or at least about 50%, or at least about 60%, or at least about 70%, or at least about 80%, or at least about 85%, or at least about 90%, or at least about 95%, or at least about 97%, or at least about 98%, or at least about 99% amino acid sequence identity therewith.

Preferably, the lipase comprising a polypeptide or peptide comprising one of the sequences of SEQ ID NO: 1-5 belongs to lipase family 1.7. The lipase comprising a polypeptide or peptide comprising the sequence of SEQ ID NO: 6 belongs to lipase family II. The lipase comprising a polypeptide or peptide comprising one of the sequences of SEQ ID NO: 7-12 belongs to lipase family VII. The lipase comprising a polypeptide or peptide comprising one of the sequences of SEQ ID NO: 13 and 14 belongs to the HSL lipase family (family IV). The lipase comprising a polypeptide or peptide comprising one of the sequences of SEQ ID NO: 15 and 16 belongs to the lipase family VIII. The lipase comprising a polypeptide or peptide comprising one of the sequences of SEQ ID NO: 17 and 18 belongs to the GDSL family. The lipase comprises a polypeptide or peptide comprising the sequences of SEQ ID NO: 19 belongs to family V.

Preferably, the lipase is an isolated lipase.

According to another aspect of the present invention, there is provided a lipase derived from bovine rumen metagenome.

According to another aspect of the present invention, there is provided a lipase derived from Anaerovibrio lipolytica 5S.

According to another aspect of the present invention, there is provided a nucleic acid sequence which encodes a lipase of the present invention or a fragment or variant thereof.

Preferably, the fragments or variants of the nucleic acid sequence of the present invention comprise a nucleic acid sequence that is hybridisable thereto under stringent conditions and/or a nucleic acid sequence that is complementary thereto.

Accordingly, in one aspect of the present invention, there is provided a nucleic acid sequence which is (i) complementary to a nucleic acid sequence which encodes a peptide or a polypeptide of the present invention; and/or (ii) hybridizable to a nucleic acid sequence which encodes a peptide or polypeptide of the present invention.

Preferably, the nucleic acid sequence is an isolated nucleic acid sequence.

Also provided by the present invention is a nucleic acid molecule comprising a nucleic acid sequence of the present invention.

Preferably, the nucleic acid molecule comprises single or double stranded DNA or RNA.

Preferably, the nucleic acid molecule further comprises vector nucleic acid sequences.

Preferably, the nucleic acid molecule further comprises nucleic acid sequences encoding a heterologous polypeptide.

Another aspect of the present invention relates to an antibody, that binds specifically with one or more lipases of the invention.

Another aspect of the present invention relates to a host cell which contains the nucleic acid molecule of the present invention.

The host cell may be a mammalian host cell or a non-mammalian host cell.

Preferably, the nucleic acid sequence is incorporated into a vector, for example a DNA plasmid. As such, in one aspect of the present invention, there is provided a vector, for example a DNA plasmid, comprising a nucleic acid sequence of the present invention.

Preferably, the plasmid is a fosmid or an open reading frame (ORF) in a plasmid.

Another aspect of the invention is a process for the preparation of a lipase of the present invention, comprising the steps of culturing an organism that expresses the lipase in a culture medium and recovering the lipase from the culture medium.

Preferably, the organism is a host cell. Preferably, the host cell may be a mammalian host cell or a non-mammalian host cell.

It is also provided a lipase obtainable by the process for the preparation of the lipase.

Preferably, the expression takes place in a suitable host cell. The host cell may be transformed with a polynucleotide or polynucleotide fragment selected from the group consisting of SEQ ID NO: 20-38, or a polynucleotide or polynucleotide fragment with sequence that has at least about 45%, or at least about 50%, or at least about 60%, or at least about 70%, or at least about 80%, or at least about 85%, or at least about 90%, or at least about 95%, or at least about 97%, or at least about 98%, or at least about 99% nucleic acid sequence identity therewith.

Preferably, the lipase obtainable from the expression of a sequence selected from the group of SEQ ID NO: 20-24 belongs to lipase family 1.7. The lipase obtainable from the expression of a sequence of SEQ ID NO: 25 belongs to lipase family II. The lipase obtainable from the expression of a sequence selected from the group of SEQ ID NO: 26-31 belongs to lipase family VII. The lipase obtainable from the expression of a sequence selected from the group of SEQ ID NO: 32-33 belongs to the lipase family IV (HSL lipase family). The lipase obtainable from the expression of a sequence selected from the group of SEQ ID NO: 34-35 belongs to lipase family VIII. The lipase obtainable from the expression of a sequence selected from the group of SEQ ID NO: 35-37 belongs to the GDSL family. The lipase obtainable from the expression of a sequence selected from the group of SEQ ID NO: 38 belongs to family V.

Another aspect of the invention is the lipase of the invention immobilized on a solid carrier material. The carrier material may be a polymer support, a resin, a macroporous resin, celite or silica gel and/or a combination thereof. The polymer support may be a polystyrene support or an acrylic support.

A further aspect of the invention is the use of the lipases of the present invention in the preparation of a polymer. The polymer may be a bioplastic. The polymer may be biocompatible. The polymer may be biodegradable. The polymer may be in the form of a film, a fibre or a nanoparticle. The polymer may be amorphous or crystalline. Preferably, the polymer comprises lactic acid subunits. The polymer may be a copolymer. The copolymer may comprise a diacid, a diol or a hydroxyl acid. The polymer may be a high molecular weight polymer.

Another aspect of the invention is a process comprising reacting one or more substrates in the presence of the lipase of the present invention. The substrate may be selected from the group of a lactide, a cyclic lactide dimmer and a low molecular weight polylactic acid oligomer. The lactide may be selected from the group of an L-lactide, a D-lactide and a DL-lactide. Preferably, the process is selected from the group of polymerization, ring opening polymerization reaction and an enzymatic copolymerization reaction. Preferably, the product of the process is a high molecular weight polymer. The polymer produced by the process may be a copolymer.

According to another aspect of the invention, there is provided a lipase formulation comprising the lipase of the present invention. The formulation may further comprise a detergent. The formulation may comprise, in addition to the lipase, stabilizers, further detergents, enzyme substrates or combinations thereof.

Another aspect of the invention is the use of the lipase of the present invention as a catalyst.

Another aspect of the invention is a process for the enzyme-catalytic conversion or enantioselective conversion of substrates comprising reacting substrates in the presence of the lipase of the present invention. Substrates of the invention may be alcohols, amines, amino acid esters, carboxylic acid esters.

Another aspect of the invention is a process for the preparation of optically active compounds comprising reacting stereoisomeric mixtures or racemates of a substrate in an enzyme catalyzed manner enantioselectively in the presence of a lipase of the present invention, and resolving the mixture.

Another aspect of the invention is a diagnostic kit comprising the lipase of the present invention. Preferably, the kit further comprises an antibody and a probe.

A further aspect of the invention is a diagnostic kit comprising an antibody binding to the lipase of the present invention.

A further aspect of the invention is a method of diagnosis comprising contacting a sample with a lipase or an antibody binding to the lipase of the present invention. The diagnosis may be for the diagnosis of blood lipid values or the presence of virulence factors.

A further aspect of the invention is the use of the lipase of the present invention in the preparation of biofuels. Preferably, the biofuel is biodiesel.

Another aspect of the invention is the use of the lipase of the present invention for oil biodegradation, waste treatment, leather degreasing.

Another aspect of the present invention relates to a peptide or polypeptide of the present invention and/or a nucleic acid sequence and/or an antibody of the present invention for use in therapy.

A further aspect of the present invention relates to a combination of two or more peptides or polypeptides of the present invention and/or a combination of two or more nucleic acid sequences and/or two or more antibodies of the present invention for use in therapy.

Another aspect of the present invention relates to use of a peptide or polypeptide of the present invention and/or a nucleic acid sequence of the present invention and/or an antibody of the invention in therapy.

A further aspect of the present invention relates to use of a combination of two or more peptides or polypeptides of the present invention and/or a combination of two or more nucleic acid sequences of the present invention and/or two or more antibodies of the present invention in therapy.

Another aspect of the present invention relates to a method for treating a patient with a disease, the method comprising administering to a patient a therapeutically effective amount of a peptide or polypeptide of the present invention and/or a nucleic acid sequence of the present invention and/or an antibody of the present invention.

Another aspect of the present invention relates to a method for treating a patient with a disease, the method comprising administering to a patient a therapeutically effective amount of a combination of two or more peptides or polypeptides of the present invention and/or a combination of two or more nucleic acid sequences of the present invention and/or a combination of two or more antibodies of the present invention.

A further aspect of the present invention relates to a method for treating a patient, the method comprising administering to a patient a therapeutically effective amount of a peptide or polypeptide of the present invention and/or a nucleic acid sequence and/or an antibody of the present invention.

A further aspect of the present invention relates to a method for treating a patient, the method comprising administering to a patient a therapeutically effective amount of a combination of two or more peptides or polypeptides of the present invention and/or a combination of two or more nucleic acid sequences of the present invention and/or a combination of two or more antibodies of the present invention.

Another aspect of the present invention relates to a composition comprising a peptide or polypeptide of the present invention and/or a nucleic acid sequence of the present invention and/or an antibody of the present invention.

A further aspect of the present invention relates to a composition comprising a combination of two or more peptides or polypeptides of the present invention and/or a combination of two or more nucleic acid sequences of the present invention and/or a combination of two or more antibodies of the present invention.

Preferably, the composition is a pharmaceutical composition.

### BRIEF DESCRIPTION OF THE FIGURES

An embodiment of the present invention will now be described by way of example, with reference to the accompanying figures in which;

FIG. 1 shows BLASTN analysis, showing that the gene sequences of lip1, lip2, lip3, lip4, lip5, lip6, lip9 and lip10 have a relatively high similarity to the gene o23 coding for an ester hydrolase in an uncultured marine prokaryote (AJ811969) (74 to 80% identity). Genes lip7, lip12 and lip14 are related (70 to 72% identity) to an esterase gene isolated from an uncultured bacterium from a cow rumen metagenome, whilst lip8 and lipll are related (75% identity) to an esterase/lipase gene retrieved from a phagemid clone from a bovine rumen metagenomic library. Phospholipase genes pll and p12 do not show any similarity to sequences documented in the GenBank database.

FIG. 2 shows the resuls of a BLASTP search on NCBI on the deduced amino acid sequences of the genes. This search showed a relatively high similarity (50 and 78 identity) between lip3, lip4, lip7, lip8, lip11, lip12 and lip14 and other esterases/lipases from uncultured rumen bacteria (ADE28720, ABI17943, CAJ19128). The esterase CA319128 is similar to that isolated from Streptococcus pyogenes (Ferrer et al., 2005b). A high similarity (63 to 84% identity) is also observed between lip1, lip2, lip5, lip6, lip9 and lip10 and the ester hydrolase from an uncultured marine prokaryote (CAH19079). Lip13 is related to a lipase from Prey. ruminicola 23 (78% identity). Phospholipases pll and p12 are related to the patatin-family phospholipase of Prey. oralis ATCC33269 (40 and 51% identity respectively). However the BLASTP search revealed that all putative proteins have a very low similarity (<40% identity) to other lipases and esterases documented in the GenBank database.

FIG. 3 shows Neighbor-joining analysis of lip1 to lip14 and lipolytic proteins from different families. The scale indicates the number of substitution events. The numbers associated with the branches refer to the bootstrap values (confidence limits) resulting from 1,000 replicate resamplings. Roman numerals correspond to the lipolytic families as defined by Arpigny and Jaeger (1999).

FIG. 4 shows the multiple amino acids alignments of lip8, lip11 and lipolytic enzymes from the HSL family.

FIG. 5 shows the conserved motifs from multiple amino acids alignments of lip1, lip2, lip5, lip6, lip9 and lip10 and lipolytic enzymes from family VII.

FIG. 6 shows the multiple alignment of lip13 and lipolytic enzymes from the GDSL family.

FIG. 7 shows the multiple amino acids alignments of lip3, lip4, lip7, lip12, lip14 and lipolytic enzymes from the subfamily 1.7.

FIG. 8 shows FT-IR trace of recovered putative polylactic acid. Lip B refers to lipase B; Lip F refers to lipase F, and N50 refers to Novozym 435.

### DETAILED DISCLOSURE OF THE INVENTION

It will of course be understood that many variations may be made to the above-described embodiment without departing from the scope of the present invention.

The invention provides lipases and for polynucleotides encoding the lipases.

In another aspect of the invention, the lipase of the present invention is used as a catalyst in a process for the preparation of a polymer. The polymer may be a bioplastic. The polymer may be biodegradable. The polymer may be polylactic acid polymer (PLA). The polymer may be an amorphous or crystalline polymer. The polymer may be moulded into parts, film, fibre and/or nanoparticles.

The PLA polymer may be prepared from a lactide, a cyclic lactide dimer, a low molecular weight polylactic acid oligomer or a lactide in combination with a copolymer. The lactide may be selected from the group of an L-lactide, a D-lactide and DL-lactide. The copolymer may be a diacid, diol or hydroxyl acid. The PLA may be prepared by a process of lipase-catalyzed ring opening polymerization.

A further aspect of the invention is a lipase as mentioned above for use in a detergent composition for use in dish washing, bleaching composition, decomposition of lipid contaminants in dry cleaning solvents, liquid leather cleaner, contact lens cleaning, cleaning of drains clogged by lipids in food processing or domestic/industrial effluent treatment plants, degradation of organic wastes on the surface of exhaust pipes, toilet bowls, removal of dirt/cattle manure from domestic animals by lipases and cellulases, washing, degreasing and water reconditioning by using lipases along with oxidoreductases, which allows for smaller amounts of surfactants and operations at low temperatures.

Another aspect of the invention is the use of a lipase as mentioned above in food processing. For example, a lipase of the invention may be used in the removal of fat from meat, including fish meat. The fat may be removed from the meat by adding a lipase during the processing of the meat. The lipase may be used in the manufacture of sausages and for determining the changes in the long-chain fatty acids liberated during the ripening of the sausages. The lipase may be used for refining rice flavour modifying soybean milk, for improving the aroma and accelerating the fermentation of apple wine. The lipase can be used for the preparation of enzyme modified cheeses. The lipase can be used for the enhancement of flavour of dairy products. Examples of such products are cheese, coffee whiteners, milk, butter, yoghurt. In this regard, the lipase can be used for accelerating the ripening of cheese products.

In another aspect of the invention, the lipase mentioned above is used in a process for the enantioselective enzyme-catalytic conversion of substrates using the lipase. Lipases as mentioned above can be used for the separation of stereoisomers and in particular for the separation of enantiomers or diastereomers from a stereoisomer mixture of the substrate. It can be used for the separation of enantiomers or diastereromers from racemic substrates and thus for the preparation for optically active compounds from the respective racemic mixtures. The resulting products can be easily separated in a manner know per se by chemical, physical and mechanical separation methods. Crystallisation, precipitation, extraction in two-phase solvent systems, chromatographic separation processes such as HPLC, GC or column chromatography on silica gel or thermal separation processes such as distillation are mentioned by way of example.

In a further aspect of the invention, the lipases as mentioned above are used for the preparation of biofuels. The term biofuel includes biodiesel fuel. Biofuels can be generated from vegetable oils.

Definitions

The following definitions shall apply throughout the specification and the appended claims.

Embodiments have been described herein in a concise way. It should be appreciated that features of these embodiments may be variously separated or combined within the invention.

Within this application, the term “lipase” is taken to mean any polypeptide, peptide or enzyme, including carboxyesterases, true lipases and phospholipases, with lipolytic activity.

Within the context of the present application, the term “comprises” is taken to mean “includes among other things”, and is not taken to mean “consists of only”.

Within this specification, the term “about” means plus or minus 20%, more preferably plus or minus 10%, even more preferably plus or minus 5%, most preferably plus or minus 2%.

As used herein, the term “functional fragments or variants thereof” means a fragment or variant of the claimed peptide or polypeptide which has lipolytic activity.

The term “isolated” means substantially separated or purified away from contaminating sequences in the cell or organism in which the nucleic acid or polypeptide naturally occurs and includes nucleic acids and polypeptides purified by standard purification techniques as well as nucleic acids prepared by recombinant technology and nucleic acids and polypeptides and polypeptide fragments chemically synthesised.

“Interact” means affecting the binding or activity of a molecule. This includes competitive binding, agonism and antagonism.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “antibody” is interpreted to mean whole antibodies and biologically functional fragments thereof. Such biologically functional fragments retain at least one antigen binding function of a corresponding full-length antibody (e.g., specificity for one or more of the peptides or polypeptides of the invention).

Within this specification, “identity,” as it is known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. Percentage identity can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988), all of which are incorporated herein by reference in their entirety. Preferred methods to determine identity are designed to give the largest match between the sequences tested. Methods to determine identity are codified in publicly available computer programs. Preferred computer program methods to determine percentage identity between two sequences include, but are not limited to, the GCG program package (Devereux, J., et al., Nucleic Acids Research 12(1): 387 (1984), which is incorporated herein by reference in its entirety), BLASTP, BLASTN, and FASTA (Atschul, S. F. et al., J. Molec. Biol. 215: 403-410 (1990), which is incorporated herein by reference in its entirety). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990), which is incorporated herein by reference in its entirety). As an illustration, by a polynucleotide having a nucleotide sequence having at least, for example, 95% “identity” to a reference nucleotide sequence of “SEQ ID NO: A” it is intended that the nucleotide sequence of the polynucleotide is identical to the reference sequence except that the polynucleotide sequence may include up to five point mutations per each 100 nucleotides of the reference nucleotide sequence of “SEQ ID NO: A.” In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having an amino acid sequence having at least, for example, 95% identity to a reference amino acid sequence of “SEQ ID NO:B” is intended that the amino acid sequence of the polypeptide is identical to the reference sequence except that the polypeptide sequence may include up to five amino acid alterations per each 100 amino acids of the reference amino acid of “SEQ ID NO: B.” In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As used herein, the term “hybridizes under stringent conditions” is intended to describe conditions for hybridization and washing under which nucleotide sequences encoding a receptor at least 50% homologous to each other typically remain hybridized to each other. The conditions can be such that sequences at least about 65%, at least about 70%, or at least about 75% or more homologous to each other typically remain hybridized to each other. Such stringent conditions are known to those skilled in the art and can be found in Current Protocols in Molecular Biology, John Wiley & Sons, N. Y. (1989), 6. 3.1-6.3.6, which is incorporated herein by reference in its entirety. One example of stringent hybridization conditions are hybridization in 6X sodium chloride/sodium citrate (SSC) at about 45° C., followed by one or more washes in 0.2 X SSC, 0.1% SDS at 50-65° C. In one embodiment, an isolated receptor nucleic acid molecule that hybridizes under stringent conditions to the sequence of SEQ ID NO:1 corresponds to a naturally occurring nucleic acid molecule.

As used herein, a “naturally-occurring” nucleic acid molecule refers to an RNA or DNA molecule having a nucleotide sequence that occurs in nature (e. g., encodes a natural protein).

As used herein, enzyme-catalytic conversions refers to chemical reactions of substrates which are catalyzed by lipases. The following reactions are examples of such reactions, but are not considered as limiting: acylation or enantioselective acylation of alcohols, acylation or enantioselective acylation of amines, acylation or enantioselective acylation of amino esters, such as, for example, amino acid esters, hydrolysis or enantioselective hydrolysis of carboxylic acid esters, ring opening polymerization.

“High molecular weight polymer” means a polymer having a molecular weight above 50,000.

“Low molecular weight polymer” means a polymer having a molecular weight between 1000 and 10000.

It will be understood that the term “biocompatible” means that the polymer is at least non toxic to cells and/or living tissues.

Within this specification embodiments have been described in a way that enables a clear and concise specification to be written, but it will be appreciated that embodiments may be variously combined or separated without parting from the invention.

The invention will now be further illustrated by the following non-limiting examples.

### EXAMPLES

Materials and Strains

All chemicals used for enzymatic tests and library screening were of the purest grade available and were purchased from Sigma-Aldrich Company Ltd. (Dorset, UK), Fisher Scientific (Leicestershire, UK) and TCI Europe (Zwijndrecht, Belgium). Restriction enzymes were from Promega UK Ltd. (Southampton, UK). Escherichia coli strains EPI300-T1R (Epicentre, Cambio Ltd., Cambridge, UK) were used for library construction and screening, and TOP10 (Invitrogen, Carlsbad, Calif., USA), for protein expression. All bacterial hosts were maintained and cultivated according to the supplier's recommendations.

Rumen Sampling and DNA Extraction

Rumen contents were collected from four rumen-fistulated, non-lactating Holstein cows (average weight of 731 kg) housed at Trawsgoed experimental farm (Aberystwyth, Ceredigion, Wales). The animals were fed a diet composed of a mixture of grass silage and straw (75:25) ad libitum and ˜1 kg of sugar beet nuts at 0700 with constant access to fresh water. Sampling was completed 2 h after concentrate feeding. Strained ruminal fluid (SRF), solid-attached bacteria (SAB) and liquid associated bacteria (LAB) were harvested as described by Huws et al. (2010).

Construction of Metagenomic Libraries

Metagenomic DNA was extracted from 200 μl of SRF, SAB and LAB using the BIO101 FastDNA® Spin Kit for Soil (Qbiogene, Cambridge, UK) following the supplier's protocol except that after the first step the samples were shaken three times for 30s, in a FastPrep bead beater (Qbiogene, Cambridge, UK) at 6 m/s with cooling on ice for 30 s between each shake. The libraries were constructed using the CopyControl™ pCC1FOS™ vector and the reagents supplied in the CopyControl™ Fosmid Library Production Kit (Epicentre, Cambio Ltd., Cambridge, UK), following the supplier's recommendations. All clones were picked using a colony picker Genetix QPix2 XT (Genetix Ltd., New Milton, England), and sub-cultured for 20 h in 384-well plates (Genetix Ltd., New Milton, England) containing LB broth with 12.5 μg/ml chloramphenicol and 20% glycerol. They were then stored at −80° C.

Screening for Lipase Activity

Selective screening of the clones for lipolytic activity was accomplished using spirit blue agar (Sigma-Aldrich Ltd., Dorset, UK) supplemented with 1% tributyrin and LB agar with 1% (w/v) trioleoyglycerol and 0.001% (w/v) rhodamine B (Kouker and Jaeger, 1987). The media were supplemented with 12.5 μg/ml chloramphenicol for selection and 2 m1/I of Copy-Control Fosmid Autoinduction solution (Epicentre, Cambio Ltd., Cambridge, UK) for high-copy number induction of the clones. The media was poured into square plates (22×22 cm) and the clones stamped onto the agar using a 384-pin replicator (Genetix Ltd., New Milton, England). After incubation for 48 h at 37oC, clones surrounded by a blue precipitate on spirit blue agar were selected. Positive clones were re-tested for lipase activity with a secondary screening, and their fosmids were extracted using the QlAprep® Spin Miniprep kit (Qiagen, Crawley, UK) following the supplier's recommendations. The fosmid size was determined after restriction with BamHI and analysis on an agarose gel.

The libraries consisted of a total of 23,872 clones: 7,744 from SRF, 8,448 from SAB and 7,680 from LAB, with an average insert size of 30-35 kbp. The libraries were screened using both a spirit blue and a trioleoylglycerol-rhodamine B assay. Five clones from the SAB library and four clones from the LAB library were positive for lipolytic/esterase activity with the spirit blue assay, while no positive clones were identified with the trioleoylglycerol-rhodamine B assay. There were no positive clones observed from the SRF library. The SAB fosmids: SAB5A16, SAB16A18, SAB16E6, SAB18J4 and SAB28M4, contained 31, 19, 31, 16 and 20 kbp of metagenomic material, respectively, whilst the LAB fosmids:, LAB4P4, LAB8M16, LAB9D24 and LAB9P23, contained of 28, 39, 33 and 18 kbp of metagenomic material, respectively.

The protein coding sequences in fosmids SAB5A16, SAB16A18, SAB16E6, SAB18J4, SAB28M4, LAB4P4, LAB8M16 and LAB9P23 were more closely related to Prevotella ruminicola 23 and Bacteroides species. Coding sequences in fosmid LAB9D24 were most closely related to Butyrivibrio fibrisolvens, Ruminococcus sp., Bacteroides sp. and Prevotella sp. (Supplementary material Table S1-S9) Fourteen putative genes showing similarity to known esterase/lipase genes were retrieved and were named lip1 to lip14, two patatin-like phospholipase genes were also found and named pl1 and p12 (Table 1). No lipase genes were retrieved from LAB8M16 or LAB9D24, either because of the incomplete assembly of the fosmid sequence due to low sequence coverage, or possibly because the blue hue observed between 20 and 24 h in the spirit blue agar plate assay was a false positive.

TABLE 1
Putative lipase/esterase genes and features of the encoded proteins
identified using a spirit blue screen of the rumen metagenome of cattle

| | | | | Protein | Theoretical |
|---|---|---|---|---|---|
| | | Length | Protein size | molecular | isoelectric |
| Fosmid | Gene | (bp) | (aa) | weight (kDa) | point |
| SAB5A16 | lip1 | 1578 | 525 | 58.86 | 4.82 |
| | lip2 | 1584 | 527 | 58.88 | 4.89 |
| | lip3 | 1749 | 582 | 65.67 | 4.63 |
| SAB16A18 | lip4 | 1743 | 580 | 65.46 | 4.53 |
| | lip5 | 1581 | 526 | 58.71 | 4.93 |
| | lip6 | 1563 | 520 | 58.28 | 4.95 |
| SAB16E6 | lip7 | 1059 | 352 | 38.93 | 5.66 |
| | lip8 | 930 | 309 | 31.79 | 5.71 |
| | pl1 | 1239 | 412 | 47.38 | 8.67 |
| SAB18J4 | lip9 | 1560 | 519 | 58.26 | 5.15 |
| | lip10 | 1677 | 558 | 62.58 | 5.32 |
| SAB28M4 | lip11 | 963 | 320 | 35.48 | 6.41 |
| | lip12 | 1086 | 361 | 40.07 | 6.34 |
| | pl2 | 2301 | 766 | 85.67 | 8.77 |
| LAB4P4 | lip13 | 846 | 281 | 31.67 | 6.26 |
| LAB9P23 | lip14 | 1059 | 352 | 38.55 | 5.14 |

DNA Sequencing and Sequence Analysis

The fosmid sequences were determined using a high throughput pyrosequencing GS FLX (454 Life Sciences) at Aberystwyth University, UK. The purified lipase-positive fosmids were fragmented to 600-900 by fragments by nebulisation. The sheared fosmids were ligated to molecular barcodes (Multiplex Identifiers, MID, Roche Life Sciences) containing short oligonucleotide adaptors “A” and “B”. This was in order to specifically tag each sample in the sequencing run. The MID-adaptor ligated DNA libraries were mixed in an equimolar amount and clonally amplified by emulsion PCR using 13.6×106 Sepharose beads per emulsion reaction. Emulsions were then broken with isopropanol and emulsion PCR beads were enriched for template-positive beads and bead-attached DNAs were denatured with NaOH and sequencing primers were annealed. Approximately 790,000 beads with clonally amplified DNA were then deposited on two of four 70×75 mm regions of the PicoTiterPlate. The PicoTiterPlate device was then loaded onto the 454 instrument along with the sequencing reagents, and sequences were obtained according to the manufacturer's protocol. An SFF (Standard Flowgram Format) file was obtained for each sample, and nucleotide sequence data and phred-like quality scores were extracted. The reads from each of the pooled libraries were identified by their MID tags by the data analysis software gsAssembler v2.5.3 (Roche Life Sciences) after the sequencing run. The assembly was done using the default parameters on gsAssembler. BLASTN on NCBI was used to trim the vector sequence from the contigs. The GC content was calculated with BioEdit. The open reading frames (ORFs) were characterized using ORF Finder (available at [http://www.ncbi.nlm.nih.gov/gorf/gorf.html]), BLASTN (non-redundant nucleotide collection) and BLASTP (non-redundant protein sequences database) on NCBI. The following criteria were applied to define ORFs:

Only sequences preferentially non-overlapping and encoding peptides longer than 50 amino acids were retained,

Where putative ORFs were found in different reading frames, only those with known homologs were retained,

In the case of various putative overlapping ORFs in different reading frames with no known homologs, the ORFs with the longest sequence were selected.

The theoretical molecular mass and isoelectric point of the deduced amino acid sequences were calculated using the Compute pI/MW tool on the ExPASy proteomics server (available at http://expasy.org/tools/pi_tool.html). Signal sequences for peptide cleavage were analyzed using SignalP 4.0 (Petersen et al., 2011) using the Gram-negative model. Conserved domains in the amino acid sequences were analyzed with Conserved-Domain search on NCBI (Marchler-Bauer et al., 2011) and the Pfam database (version 25.0, available at http://pfam.sanger.ac.uk/). Clustering analysis was conducted by carrying out multiple sequence alignments using the ClustalW online tool (available at http://www.ebi.ac.uk/Tools/msa/clustalw2/) for the protein sequences. Closely related homologs were identified from the NCBI non-redundant database using BLASTP searches. Sequences with alignment >50% identities and e-value <1e-10 were considered. As the classification of lipolytic enzymes is based on the comparison of their protein sequences (Arpigny and Jaeger, 1999, Haussmann and Jaeger, 2010), the protein sequences from 50 members representing eight lipolytic families were retrieved from NCBI (Liu et al., 2009). A multiple sequence alignment file was constructed using ClustalW online tool on the Pfam conserved domains (α/β hydrolase fold or esterase/lipase domain). MEGA5 software (Tamura et al., 2011) was used to construct the tree using the neighbor-joining method by following Dayhoff PAM matrix model.

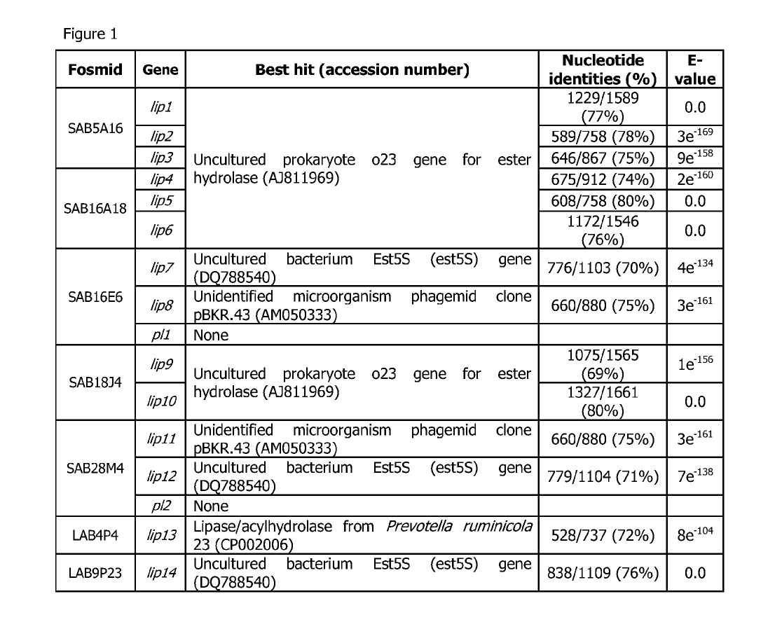

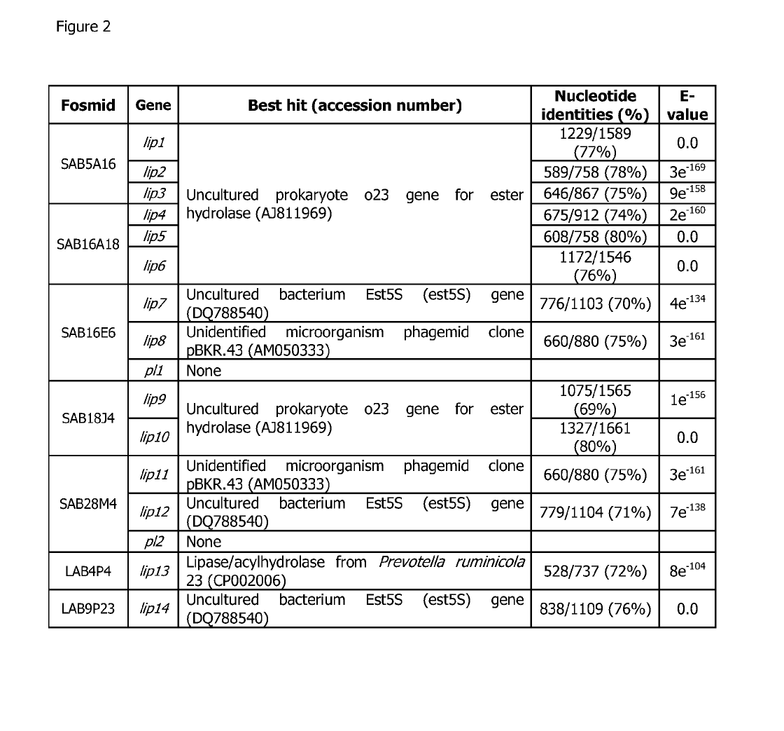

BLASTN analysis showed that the gene sequences of lip1, lip2, lip3, lip4, lip5, lip6, lip9 and lip10 had some similarity to the gene o23 coding for an ester hydrolase in an uncultured marine prokaryote (AJ811969) (74 to 80% sequence identity). Genes lip7, lip12 and lip14 were related (70-72% sequence identity) to an esterase gene isolated from an uncultured bacterium from a cow rumen metagenome, whilst lip8 and lip11 were related (75% sequence identity) to an esterase/lipase gene retrieved from a phagemid clone from a bovine rumen metagenomic library (FIG. 1). Phospholipase genes pl1 and pl2 did not show any similarity to sequences documented in Genbank. The deduced amino-acid sequences of the genes were used to perform a BLASTP search against the NCBI database. A relatively high similarity (between 50 and 78% identity) was observed between lip3, lip4, lip7, lip8, lip11, lip12 and lip14 and other esterases/lipases from uncultured rumen bacteria (ADE28720, ABI17943, CAJ19128). Ferrer et al., (2005b) have previously reported that CAJ19128 was similar to an esterase isolated from Streptococcus pyogenes. A high similarity (63 to 84% identity) was also observed between lip1, lip2, lip5, lip6, lip9 and lip10 and the ester hydrolase from an uncultured marine prokaryote (CAH19079). Lip13 was related to a lipase from P. ruminicola 23 (78% identity). Phospholipases pl1 and pl2 were related to the patatin-family phospholipase of Prevotella oralis ATCC33269 (40 and 51% identity respectively) (FIG. 2). However a BLAST search revealed that all putative proteins had a very low similarity (<40% identities) to other lipases and esterases documented in GenBank.

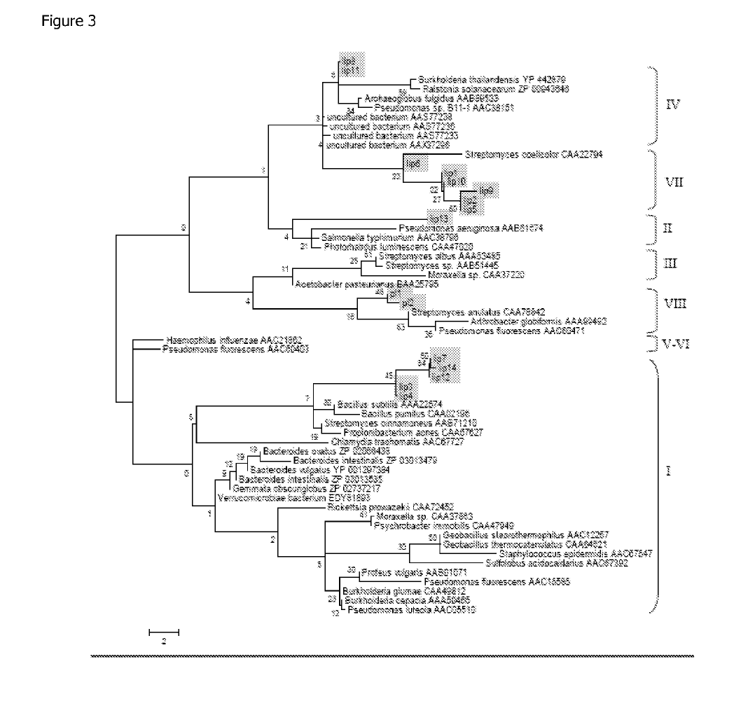

Phylogenetic placement of the predicted proteins suggest that lipases from all the main lipase families described by Arpigny and Jaeger (1999), shown in FIG. 3, had been recovered, however as the bootstrap values were low further analysis was conducted separately for putative esterases and lipases. Domain analysis (FIG. 2) revealed that lip1, lip2, lip5, lip6, lip9 and lip10 contained domains linked to lipase and esterase activity (esterase/lipase superfamily domain, carboxylesterase domain) and their catalytic triads were predicted. Lip8 and lip11 contained an gip hydrolase fold domain, whilst lip3, lip4, lip7, lip12 and lip14 had only a DUF3089 domain, which represents an a/13 hydrolase fold and therefore putative enzymatic activity. Lip13 contained a rhamnogalacturonan esterase like domain. PI1 and pI2 were predicted to be outer membrane proteins, pl2 also contained a patatin-like phospholipase domain and a domain predicted to code for an esterase of the a/13 hydrolase family. Proteins lip3, lip4, lip7, lip10, lip12, lip13, lip14 and pI2 were predicted to be secreted enzymes based on the presence of a putative signal peptide.

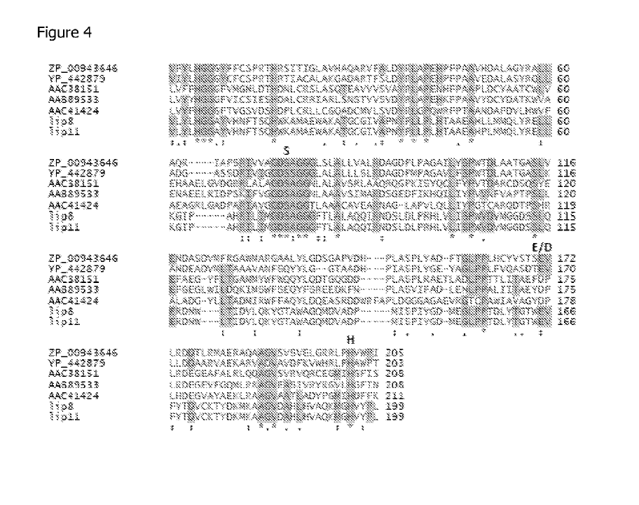

Lip8 and lip11 clustered with genes from family IV as defined by Arpigny and Jaeger (1999). This was confirmed by multiple alignments with proteins from this family (FIG. 4), lip8 and lip11 contained the lipase-conserved catalytic triad of residues Glu, replacing Asp (residue 229 and 263 for lip8 and lip11 respectively), His (259, 293 respectively) and the catalytic nucleophile Ser (138, 172), respectively, in the consensus pentapeptide GDSAG. The HSL family conserved HGGG motif, amino acids 69 to 71 and 103 to 105, respectively, was found upstream of the active-site conserved motif.

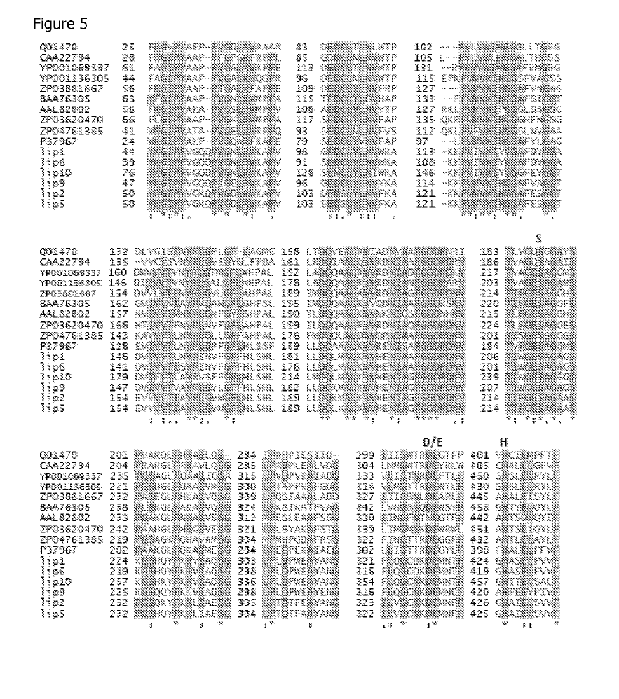

Alignments indicated that lip1, lip2, lip5, lip6, lip9, and lip10 might be more closely related to family VII. Multiple amino acid alignment (FIG. 5) with enzymes related to this family confirmed it: the catalytic triad was present with Asp, His and Ser in the consensus motif GESAG.

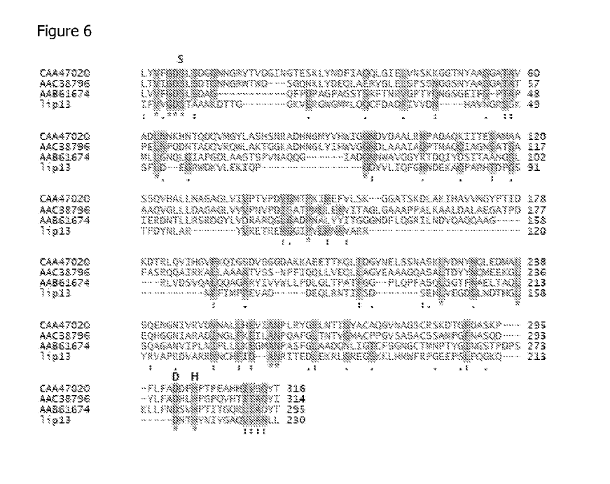

Lip13 clustered with so-called GDSL enzymes from family II. The active site motif GDS(L) was found in the N-terminus of the protein sequence and elements of the five blocks of conserved amino-acids were present in its sequence (FIG. 6).

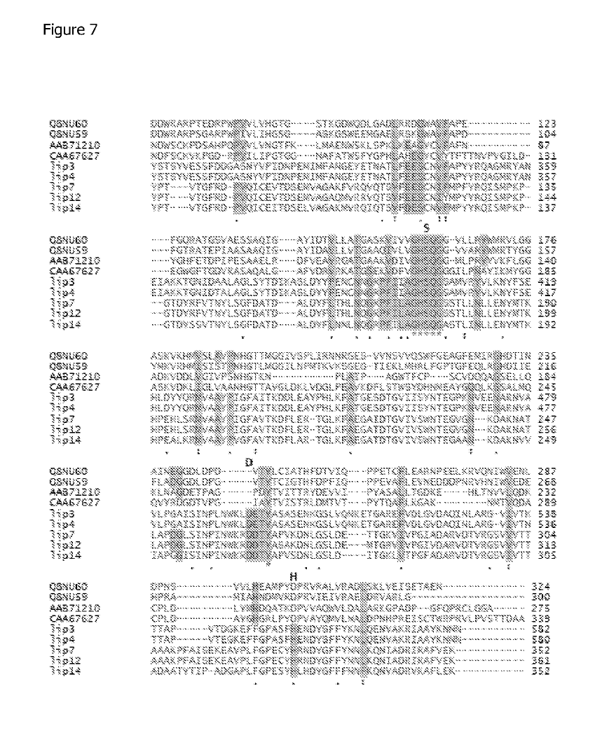

The dendrogram (FIG. 3) suggested that lip3, lip4, lip7, lip12 and lip14 clustered with true lipases from subfamily 1.7. Multiple amino acid alignments with lipases included in this family showed that the proteins contained the conserved motif GHSQG (FIG. 7). However the alignments did not show other conserved blocks and the putative catalytic triad was not identified for lip3, lip4 and lip14 with the Asp missing.

Pl1 and pl2 clustered with enzymes from family VIII; however, multiple amino acid alignments did not show conserved motifs characteristic from this enzyme family.

Preparation of Anaerovibrio Lipolytica 5S Genomic DNA

Pure cultures of A. lipolytica strain 5S, as first isolated by Hobson and Mann (1961) at the Rowett Research Institute (Aberdeen, Scotland), came from the Rumen Microbiology group collection at IBERS. The genomic DNA was extracted using the BIO101 FastDNAC) Spin Kit for Soil (Qbiogene, Cambridge, UK) from approximately 2 mg of cryopreserved freeze-dried culture. The manufacturer's guidelines were followed, with the exception that the sample was processed for 3×30 s at speed 6.0 in the FastPrep instrument (QBiogene), with incubation for 30 s on ice between bead-beating.

De novo Genome Sequencing

The draft nucleotide sequence of the bacterium was established by a shotgun sequencing approach carried out on a Genome Sequencer FLX system (454 Life Sciences, Roche), following the supplier's protocol. Assembly of the reads was accomplished using gsAssembler v2.5.3 software (Roche, Life Sciences), using the default parameters.

454 pyrosequencing generated 340,862 high quality reads with an average length of 425.16 bp, representing 144,706,594 by total information. These data represented 36×coverage for an estimated bacterial genome size of 4 Mbp. The assembly of the uncompleted draft genome resulted in 285 contigs with 2,830,874 by total sequence information, comprising 247 large contigs (>500 bp) with a total size of 2,816,384 bases. The RAST annotation identified 2,673 coding sequences and the G+C content was 43.28%. Copies of the 5S and 23S rRNA genes (6 and 1 respectively) and 60 predicted tRNA genes were identified within the genome. There are 268 subsystems represented in the genome, however 63% of the predicted genes could not be assigned to a subsystem. Two genes annotated as “GDSL family lipolytic enzyme” and one gene annotated as “carboxylesterase” were named alipA, alipB and alipC respectively.

Annotation and Sequence Analysis

The contigs were submitted for genome annotation to the RAST server at http://rast.nmpdr.org (Aziz et al., 2008), tRNAscan-SE 1.23 (Lowe and Eddy, 1997) and RNAmmer 1.2 (Lagesen et al., 2007).

The predicted lipase genes and amino acid sequences were compared for similarity to known sequences using BLASTN and BLASTP search. Their signal sequences for peptide cleavage were predicted using SignalP 4.0 (Petersen et al., 2011). CD search (Marchler-Bauer et al., 2011), the Pfam database (version 25.0, available at http://pfam.sanger.ac.uk/) and ClustalW (Thompson et al., 1994) were used to search for conserved domains in the predicted amino acid sequences and to execute multiple alignments to find potential gene products relatedness to known families of lipolytic enzymes. The theoretical molecular mass and isoelectric point of the deduced lipolytic protein sequences were calculated using the Compute pI/Mw tool on the ExPASy proteomics server (available at http://expasy.org/tools/pi_tool.html, May 2011).

The genes length varied from 744 to 1,476 bp; and features of the encoded proteins are presented in Table 2. Tables 3 and 4 present the results of the BLASTN and BLASTP analysis of the identified putative lipase genes.

Gene alipA matched with a gene coding for a lipolytic protein from Selenomonas sputigena, with 63% identity, whereas no homologous sequences were found for genes alipB and alipC in the Genbank database. However, the proteins were found to match with proteins from various Veillonellaceae, and the best hits were with GDSL lipolytic proteins from Selenomonas species for the proteins alipA and alipB (56 and 41% identity respectively) and with a lipase/esterase from Mitsuokella multiacida for alipC (51% identity). AlipC also shared 42% amino acid identity (e-value 8e-50) with the lipase from rumen metagenome RlipE2 (Liu et al., 2009).

TABLE 2
Putative lipase/esterase genes identified from Anaerovibrio lipolytica
5S using the RAST annotation and features of the encoded proteins

| | | | Protein | |
|---|---|---|---|---|
| | | | molecular | Theoretical |
| Gene | Length (bp) | Protein size (aa) | weight (kDa) | isoelectric point |
| alipA | 1476 | 492 | 56.271 | 5.64 |
| alipB | 1314 | 438 | 48.795 | 4.94 |
| alipC | 744 | 248 | 27.750 | 6.17 |

TABLE 3
Best matches obtained using BLASTN for the lipolytic genes identified in
Anaerovibrio lipolytica 5S

| | | | Nucleotide | |
|---|---|---|---|---|
| | | | identities | E- |
| Contig | Gene | Best hit (accession number) | (%) | value |
| Contig00046 | alipA | Lipolytic protein GDSL family | 533/842 | 2e⁻¹³ |
| | | (CP002637) from Selenomonas | (63%) | |
| | | sputigena ATCC 35185 | | |
| Contig00136 | alipB | None | — | — |
| Contig00239 | alipC | None | — | — |

TABLE 4
Best matches using BLASTP for the predicted amino acid sequence
of lipolytic genes identified in Anaerovibrio lipolytica 5S

| | | | Identity |
|---|---|---|---|
| | Putative function | Most similar | (overlapped |
| Protein | (accession number) | homolog (e-value) | aa) |
| alipA | Lipolytic protein | Selenomonas sputigena | 278/495 (56%) |
| | GDSL family | ATCC 35185 (0.0) | |
| | (AEC00120) | | |
| alipB | GDSL-like protein | Selenomonas sp. oral | 169/411 (41%) |
| | (EFR39963) | taxon 137 str. | |
| | | F0430 (5e⁻¹⁰⁸) | |
| alipC | Putative esterase/ | Mitsuokella multiacida | 126/249 (51%) |
| | lipase (EEX68534) | DSM 20544 (3e⁻⁸⁰) | |

Expression and Purification of Recombinant Lipases

Primers for the amplification of the lipase genes were designed with FastPCR 6.1 (Kalendar et al., 2009), with and without the N-terminal signal sequence where one could be identified. The PCR reaction was set up in a total volume of 25 μl as follows: 2 μL of template (˜100 ng), 1 μl of forward and reverse primer (10 μM), 8.5 μl of molecular water and 12.5 μl of PCR mastermix (ImmoMix™, Bioline UK Ltd., London, UK). Initial activation of the Taq was performed for 10 min at 95° C., followed by 25 cycles as follows: 95° C. for 30 s, 50° C. for 30 s, 72° for 2 min, followed by a final extension at 72° C. for 8 min and holding of samples at 4° C. After PCR, the products were verified by electrophoresis on a 1% agarose gel using a 1 kb ladder. The band of interest was cut out with a sterile razor blade and the DNA eluted using the MinElute Gel Extraction kit (Qiagen, Crawley, UK).

TABLE 5
Primers used for amplification of the lipase genes in the lipase positive
fosmid clones and Anaerovibrio lipolytica 5S

| | Gene | | | SEQ ID | Expected size |
|---|---|---|---|---|---|
| Template | amplified | Primer | Sequence (5′-3′) | NO. | of product (bp) |
| SAB5A16 | lip1 | lip1 F | gattgggaga gaacctattt cctc | 57 | 1626 |
| | | lip1 R | ccgtcaaggc atataatagt tggt | 58 | |
| | lip2 | lip2 F | actatggatg ctcaagagat taga | 59 | 1587 |
| | | lip2 R | ttaaagcata taatatttgg tgaggaa | 60 | |
| | lip3 | lip3 F | atgagacaat tgaaaaagtg gatgctt | 39 | 1773/1701(ss) |
| | | lip3 ssF | tcgaacgatg ataattcctc c | 41 | |
| | | lip3 R | agcatccata gttttgtttc cttt | 40 | |
| SAB16A18 | lip4 | lip4 F | atgagacaat tgaaaaagtg gatg | 42 | 1773/1710(ss) |
| | | lip4 ssF | gcgtgctcgt cgaacgaaga c | 43 | |
| | | lip4 R | ctcttgagca tccatagttt tgtt | 44 | |
| | lip5 | lip5 F | actatggatg ctcaagagat taga | 61 | 1620 |
| | | lip5 R | cgtattgttc catattcgtt ttca | 62 | |
| | lip6 | lip5b F | ggagaggacc tacttcctca c | 63 | 1665 |
| | | lip5b R | agcgagccca attggtgtga t | 64 | |
| SAB16E6 | lip7 | lip6 F | aatatgaaaa gacagaattt cctcgtg | 45 | 1065/999(ss) |
| | | lip6 ssF | tcgtgcaaga gtagtaacaa a | 46 | |
| | | lip6 R | ttctcacttt tccacgaacg c | 47 | |
| | lip8 | lip6b F | agagtggctg cggtaatcat c | 70 | 978 |
| | | lip6b R | gcccgacgta agtattccta t | 71 | |
| | pl1 | pl1 F | ccactgcgac cgaatgccat g | 74 | 1323 |
| | | pl1 R | cctccgttga atctgtcgat t | 75 | |
| SAB1834 | lip9 | lip7 F | ggctgttcct gttacacata a | 65 | 1623 |
| | | lip7 R | ggtaacttga acctgtcaaa g | 66 | |
| | lip10 | lip8 F | atgagaaatt ttaaaaagtg gatgcttgcc | 67 | 1695/1632(ss) |
| | | lip8 ssF | acttcatgca gtagcaaaga agac | 68 | |
| | | lip8 R | agtgatcgcg gaaaacagtc a | 69 | |
| SAB28M4 | lip11 | lip9 F | gcgttcgtgg aaaagtgaga a | 72 | 1044 |
| | | lip9 R | gcccgacgta agtattccta t | 73 | |
| | lipl2 | lip10 F | tccggttgat gaagaaatat gcaa | 48 | 1107/999(ss) |
| | | lip10 ssF | tcgtgcaaga gcagtaacaa accc | 49 | |
| | | lip10 R | ttctcacttt tccacgaacg c | 50 | |
| | p12 | p12 F | gacgcccctg gaatatagaa ttaa | 76 | 2340/2247(ss) |
| | | p12 ssF | gctaagtcag agaaagaggc t | 77 | |
| | | p12 R | ttagaattca tatccgaggt tgat | 78 | |
| LAB4P4 | lip13 | lip11 F | atgatgagaa gtctgaaggt t | 54 | 849/789 (ss) |
| | | lipll ssF | gccacacccg acaagaccac t | 55 | |
| | | lip11 R | aacttatttt gcgttccgaa c | 56 | |
| LAB9P23 | lip14 | lip12 F | gccaggctac tggacaagaa t | 51 | 1101/993(ss) |
| | | lip12 ssF | tcgtgcaaga tgagtaacaa a | 52 | |
| | | lip12 R | ttgttatttc tccaggaatg c | 53 | |
| Anerovibrio | alipA | alipA F | accatggatt ggacccgtta t | 79 | 1482 |
| lipolytic | | alipA R | tgcttattgc ttaaatactt ccttgtg | 80 | |
| 5S | alipB | alipB F | gggagagata acatgaagtt ttcc | 81 | 1344/1263(ss) |
| | | alipB ssF | gcagaggata ttaatactga g | 82 | |
| | | alipB R | ttcgtgagac agcttttttt a | 83 | |
| | alipC | alipB F | accagcatga ttattaacgg c | 84 | 855 |
| | | alipB R | caccggctct ctatgtgtta t | 85 | |

F, forward primer; ssF, forward primer with predicted signal sequence removed; R, reverse primer

TABLE 6
Relative positions of the primers in regard to the start and stop codons
of the lipolytic genes

Position of the 5′ end of the primers

| | Forward primer | Reverse primer |
|---|---|---|
| | Number of codons before | Number of codons after |
| Gene | the first Met | the stop codon |
| lip1 | 7 | 7 |
| lip2 | 1 | — |
| lip3 | — | 8 |
| lip4 | — | 10 |
| lip5 | 1 | 12 |
| lip6 | 21 | 13 |
| lip7 | 1 | 1 |
| lip8 | 1 | 15 |
| lip9 | 5 | 16 |
| lip10 | 6 | — |
| lip11 | 15 | 12 |
| lip12 | 1 | 6 |
| lip13 | — | 1 |
| lip14 | 1 | 13 |
| pl1 | 6 | 22 |
| pl2 | — | 13 |
| alipA | 1 | 1 |
| alipB | 5 | 4 |
| alipC | 2 | 2 |

The expression of the lipolytic genes was then undertaken using the pTrcHis TOPO® TA Expression kit (Invitrogen, Carlsbad, Calif., USA) following the supplier's protocol. The PCR product was ligated to the pTrcHis TOPO vector and transformed into E. coli TOP10 cells. Twelve colonies for each transformation were picked for secondary screening and their insert was analyzed for size and orientation by tip-dip PCR using the gene specific forward primer and the vector specific pTrcHis reverse primer (5′-GATTTAATCTGTATCAGG-3′).

Proteins from the rumen metagenome were purified from 50 ml cultures in the presence of 50 μg/ml ampicillin on 2% inoculation from 2 ml of LB starter culture containing 50 μg/ml ampicillin (inoculated with a single colony) and grown overnight at 37° C. with shaking. The culture was induced with 1 mM IPTG after growth to mid-log growth phase and grown with shaking for a further 5 h at 37° C. The cells were then harvested by centrifugation at 3000×g, 10 min, 4° C., and the pellets stored at -80° C. before proceeding to protein purification. Purification of the proteins was carried out in native conditions using the ProBond™ Purification System (Invitrogen, Carlsbad, Calif., USA). Protein concentrations were estimated using the Bradford procedure (Bradford, 1976) employing BSA as the standard (Sigma, Dorset, UK).

Protein expression of A. Lipolytica lipases was accomplished by growing and inducing 50 ml of cells as follows: 2 ml of LB broth containing 50 μg/ml ampicillin were inoculated with a single colony and grown overnight at 37° C. with shaking. Subsequently, 50 ml of LB broth containing 50 μg·ml-1 ampicillin were inoculated with 1 ml of the overnight culture and grown until mid-log. The culture was then induced with IPTG to a final concentration of 1 mM and the culture grown at 37° C. with shaking at 100 rpm for 5 h. The cells were then harvested by centrifugation at 3000×g, 10 min, 4° C., and the pellets stored at -80° C. before proceeding to protein purification. Purification of the proteins was carried out in native conditions using the ProBond™ Purification System (Invitrogen, Carlsbad, Calif., USA). Sodium dodecyl sulfate-polyacrylamide gel electrophoresis (SDS-PAGE) was used to examine the success of the purification.

Protein concentration was estimated using the Bradford procedure (Bradford, 1976) employing BSA as the standard (Sigma, Dorset, UK). The enzyme sample (5 μl) was mixed with 250 μl of Bradford reagent in a microplate, the plate was shaken for 30 s and incubated at room temperature for 20 min. The formation of the blue-coloured Coomassie-Blue G-250 complex was then monitored at 595 nm on a PowerWave XS microplate reader (BioTek Instruments Inc., Potton, UK).

Enzymatic Assays

Enzyme activity was quantified on a temperature-controlled Powerwave XS microplate reader (BioTek Instruments Inc., Patton, UK) based on the level of ρ-nitrophenol released following the hydrolysis of ρ-nitrophenyl ester substrates by the enzyme (Lee et al., 1993; Pinsirodom and Parkin, 2001). The production of ρ-nitrophenol was monitored in triplicates every minute for 10 min at 410 nm, and data were collected with the software Gen5 v1.10 (BioTek Instruments Inc., Potton, UK). Unless otherwise described, enzyme activity was measured by a standard assay at 39° C., with 1 mM ρ-nitrophenyl ester substrates in 50 mM morpholineethanesulfonic acid (MES, pH 6.5) containing 1% acetonitrile. The substrates used in standard conditions were ρ-nitrophenyl caprate (C10) for lip4 and lipl3ss, ρ-nitrophenyl caproate (C6) for lip6 and ρ-nitrophenyl caprylate (C8) for pl1 and pl2ss, alipA, alipBss and alipC. After pre-incubation for 3 min, the reaction was started by the addition of 2 μl of the eluted fraction of purified enzyme (˜0.4 mg/ml). Blank reactions were performed with every measurement to subtract appropriate values for nonenzymatic hydrolysis of the substrate. One unit of enzyme activity was defined as the amount of activity required to release 1 pmol of ρ-nitrophenol/min from ρ-nitrophenyl ester.

Substrate Specificity

The following ρ-nitrophenyl esters with different chain length were used at 1 mM final concentration for assaying substrate specificities: ρ-nitrophenyl butyrate (C4), ρ-nitrophenyl caproate (C6), ρ-nitrophenyl caprylate (C8), ρ-nitrophenyl caprate (C10), ρ-nitrophenyl laurate (C12), ρ-nitrophenyl myristate (C14), ρ-nitrophenyl palmitate (C16) and p-nitrophenyl stearate (C18). The ρ-nitrophenyl ester substrates with C4 to C10 acyl chains were dissolved in acetonitrile at a concentration of 100 mM. P-nitrophenyl ester substrates with C12 to C18 acyl chains were dissolved in a 1:4 mixture of acetonitrile and 2-propanol in order to solubilise the substrate, and reactions were performed with final concentrations of 1% acetonitrile and 4% 2-propanol.

To examine substrate specificity, activity was tested against various ρ-nitrophenyl esters with different acyl chain lengths. The results under standard assay conditions of pH 6.5 and 39° C. are presented in Table 7.

Lip4 and lipl3ss showed narrow chain length specificity, with the highest specific activity against ρ-nitrophenyl laurate (373.4 and 398.6 U/mg respectively) and a lower specific activity against ρ-nitrophenyl caprate (107.7 and 214.6 U/mg respectively); activity against other substrates was very low or not detected.

Protein lip6 exhibited the typical behavior for carboxylesterases, showing a preference for short acyl chains. The highest specific activity was observed with ρ-nitrophenyl butyrate (273.3 U/mg) and activity values decreased with the increase in the acyl chain length. No detectable activity was observed against ρ-nitrophenyl stearate.

Pl1 showed a broader range of activity with higher specific activities against short to medium acyl chain length: the activities were respectively 247.8 U/mg with ρ-nitrophenyl butyrate, 317.5 U/mg with ρ-nitrophenyl caprylate, 224.6 U/mg with ρ-nitrophenyl caprate and laurate.

Pl2ss showed no identifiable substrate preference. AlipA and alipBss showed a narrow chain length specificity, with the highest specific activity against ρ-nitrophenyl laurate (640 U·mg-1) and myristate (157 U·mg-1) respectively, and lower specific activity against ρ-nitrophenyl caprate (33 and 43 U·mg-1 respectively). AlipC showed a broader range of activity with higher specific activities against short to medium acyl chain length: the activities were 187 U·mg-1 against ρ-nitrophenyl butyrate, 270 U·mg-1 against ρ-nitrophenyl caprylate, 118 and 242 U·mg-1 against ρ-nitrophenyl laurate.

TABLE 7
Substrate specificity of lipases isolated from the rumen metagenome of cattle and from A. Lipolytica

| | Specific activity (U/mg protein) | | | | | | | |
|---|---|---|---|---|---|---|---|---|
| Substrate | lip4 | lip6 | lip13ss | pl1 | pl2ss | alipA | alipB | alipC |
| pNP-acylesters | | | | | | | | |
| Butyrate (C4) | 56.3 ± 12.1 | 273.3 ± 22.5 | ND | 247.8 ± 11.1 | 172.5 ± 12.0 | 139.4 ± 17.4 | 91.8 ± 22.9 | 186.7 ± 41.5 |
| Caproate (C6) | 28.7 ± 24.9 | 198.6 ± 10.8 | 51.1 ± 14.5 | 154.9 ± 21.8 | 58.8 ± 20.4 | 73.8 ± 19.0 | 48.6 ± 11.5 | 76.0 ± 48.9 |
| Caprylate (C8) | 36.0 ± 23.7 | 42.4 ± 16.7 | 20.5 ± 14.4 | 317.5 ± 31.6 | 141.2 ± 17.0 | 98.4 ± 17.4 | 59.4 ± 21.8 | 269.6 ± 71.7 |
| Caprate (C10) | 107.7 ± 37.3 | 30.5 ± 5.1 | 214.6 ± 14.5 | 224.6 ± 5.5 | 109.8 ± 4.5 | 32.8 ± 9.5 | 43.2 ± 11.4 | 117.5 ± 5.3 |
| Laurate (C12) | 373.4 ± 45.7 | 23.8 ± 8.7 | 398.6 ± 7.1 | 224.6 ± 11.0 | 274.5 ± 36.3 | 639.6 ± 24.0 | 97.2 ± 24.7 | 242.0 ± 38.4 |
| Myristate (C14) | 71.7 ± 12.4 | 18.7 ± 4.3 | 153.3 ± 25.6 | 209.1 ± 20.4 | 227.4 ± 27.2 | 172.2 ± 17.4 | 156.6 ± 11.5 | 193.6 ± 34.2 |
| Palmitate (C16) | ND | 13.6 ± 7.0 | ND | 162.6 ± 32.4 | 235.3 ± 67.9 | 123.0 ± 19.1 | 64.8 ± 27.6 | 83.0 ± 37.5 |
| Stearate (C18) | ND | ND | ND | 46.5 ± 32.8 | 109.8 ± 29.7 | 73.8 ± 17.3 | NDetect | 34.6 ± 20.8 |
| Triglycerides | | | | | | | | |
| Tributyrin (C4) | 55.8 | 51.6 | 26.5 | 130.0 | 65.8 | ND | ND | ND |
| Tricaprylin (C8) | 55.8 | 56.8 | 26.5 | 65.0 | 131.7 | ND | ND | ND |
| Triolein (C18:1) | 55.8 | ND | ND | ND | 131.7 | ND | ND | ND |

ND, not determined
NDetect, not detected

Effect of pH on Enzyme Activity

The effect of pH on the activity of the enzymes was examined across the pH range 3.5 to 10.0 using a wide-range pH buffer (Cai et al., 2011), containing 40 mM each of acetic acid, MES, N-(2-hydroxyethyl) piperazine-N′-ethanesulfonic acid (HEPES), N-[Tris(hydroxymethyl) methyl]-3-ami nopropanesulfonic sodium salt (TAPS) and N-cyclohexyl-3-aminopropane sulfonic acid (CAPS). The pH was adjusted by adding 1 M HCl or 1 M NaOH as appropriate at 39° C. The specific activity of the enzyme was determined spectrophotometrically at 348 nm as it is the pH-independent isobestic wavelength of ρ-nitrophenoxide and ρ-nitrophenol (Hotta et al., 2002).

The pH assays were carried out using ρ-nitrophenyl caprate (C10) as the substrate for lip4 and lipl3ss, ρ-nitrophenyl caproate (C6) for lip6 and ρ-nitrophenyl caprylate (C8) for pl1 and pl2ss, at a constant temperature of 39° C. in a wide-range pH buffer set at the indicated pH values.

Proteins Lip4, lip6, lipl3ss and pl1 had maximal activity at neutral or slightly alkaline pH (7 or 7.5). Lip6, lipl3ss and pl1 exhibited >50% activity in the pH range of 6.5-8.0, while lip4 showed activity over a broader pH range as it presented 53% of its maximum activity level at pH 10. Pl2ss had optimum pH at 8.5 respectively and presented activity >50% in alkaline pH range 8.5-10.0.

AlipA and alipC had maximal activity at pH 8.5 and 9.0 respectively, and presented >50% activity in alkaline pH ranges, respectively 7.5-9.5 and 9.0-10.0. AlipBss showed >50% of maximum activity in the pH range 6.0-8.0, with maximal activity at pH 7.5.

Effect of Temperature on Enzyme Stability and Thermostability

The effect of temperature on the activity of enzyme activity was examined across the range 25-70° C. under standard assay conditions. The pH of the MES buffer was adjusted to 6.5 at respective temperatures. The thermostability of the enzymes was analyzed by measuring the residual activity after incubating the enzyme (2 μl in 50 mM MES, pH 6.5) for 1 h at 50, 60 and 70° C.

The assays were carried out using ρ-nitrophenyl caprate (C10) as the substrate for lip4 and lipl3ss, ρ-nitrophenyl caproate (C6) for lip6 and ρ-nitrophenyl caprylate (C8) for pl1 and pl2ss, at a constant pH of 6.5 in 50 mM MES.

The optimum temperatures were determined as 40° C. (lip4, lip6, lipl3ss), 45° C. (pl1), and 30° C. (pl2ss). The temperature range where the enzyme retained more than 50% activity was narrow for lip4 (around 40° C.), pl1 (45-50° C.) and lipl3ss (around 40° C.), while it was broader for lip6 (40-50° C.) and pl2ss (25-40° C.). The temperature stability of the proteins was examined by measuring residual activity after incubating the purified enzymes for 1 h at 50, 60 or 70° C. as shown in Table 8. The proteins lip4, lipl3ss, and pl1 appeared to be temperature sensitive as less than 50% of activity was measured after 1 h incubation at 50° C. Activities ranged from 8 to 45% after incubating at 60 or 70° C. Lip6 appeared to have some thermostability: at 50° C., it had 57.9% activity but lost activity after incubation at 60 or 70° C. Only the protein pl2ss displayed some thermostability as it displayed nearly 90% of its activity after incubation at 50 and 60° C., yet lost 30% of its activity after incubation at 70° C.

The optimum temperatures were determined as 40° C. (alipA, alipC) and 55° C. (alipBss). The temperature range where the enzyme retained more than 50% activity was 40-50° C. for alipA, 35-55° C. for alipBss, and 35-50° C. for alipC. The temperature stability of the proteins was examined by measuring its residual activity after incubating the purified enzymes for 1 h at 50, 60 or 70° C. (Table 8). The proteins alipBss and alipC appeared to be temperature sensitive as less than 50% of activity was measured after 1 h incubation at 50° C. Activities ranged from 8 to 45% after incubating at 60 or 70° C. AlipA appeared to have some thermostability: it retained around 50% activity after incubation at 60 and 70° C.

TABLE 8
Relative activity of lipases isolated from the rumen metagenome of cattle after incubation for 1 h at 50, 60 or 70° C.

| Temperature | Relative activity (%) | | | | | | | |
|---|---|---|---|---|---|---|---|---|
| of incubation | lip4 | lip6 | lip13ss | pl1 | pl2ss | alipA | alipB | alipC |
| 40° C. | 100 | 100.0 | 100.0 | 100.0 | 100.0 | 100.0 | 100.0 | 100.0 |
| 50° C. | 41.8 ± 5.6 | 57.9 ± 4.3 | 25.7 ± 11.5 | 37.0 ± 10.5 | 87.8 ± 10.3 | 16.8 ± 9.8 | 33.6 ± 11.0 | 32.8 ± 12.4 |
| 60° C. | 47.5 ± 8.1 | 11.5 ± 7.8 | 10.5 ± 18.7 | 15.1 ± 7.0 | 89.6 ± 13.2 | 45.3 ± 12.5 | 24.0 ± 6.1 | 8.2 ± 5.8 |
| 70° C. | 31.3 ± 15.5 | 13.0 ± 4.3 | 11.4 ± 3.4 | 16.5 ± 7.0 | 73.8 ± 10.9 | 49.2 ± 8.4 | 15.6 ± 6.5 | 9.8 ± 3.2 |

Effect of Metal Ions on Enzyme Activity

The effect of metal ions on the activity of the enzymes were investigated by incubating the enzymes with various metal chloride salts (Na⁺, K⁺, NH⁴⁺, Mg²⁺, Ca²⁺, Mn²⁺, Zn²⁺, Co²⁺) at final concentrations of 5 mM in 50 mM MES (pH 6.5) for 30 min at room temperature. The remaining activity was then measured under standard assay conditions. The results are presented in Table 9.

TABLE 9
Effect of metal ions on the relative activity of lipases isolated from the bovine rumen metagenome

| | Relative activity (%) | | | | | | | |
|---|---|---|---|---|---|---|---|---|
| Ions | lip4 | lip6 | lip13ss | pl1 | pl2ss | alipA | alipB | alipC |
| unincubated | 100.0 | 100.0 | 100.0 | 100.0 | 100.0 | 100.0 | 100.0 | 100.0 |
| Na⁺ | 69.8 ± 1.8 | 101.2 ± 2.4 | 75.9 ± 1.5 | 76.2 ± 1.9 | 119.2 ± 23.0 | 107.3 ± 2.5 | 71.6 ± 5.7 | 119.4 ± 5.4 |
| K⁺ | 88.4 ± 1.8 | 16.9 ± 0.5 | 49.8 ± 2.0 | 76.2 ± 1.5 | 260.2 ± 30.7 | 98.3 ± 4.0 | 67.1 ± 1.6 | 100.5 ± 3.6 |
| NH₄⁺ | 51.2 ± 1.6 | 50.6 ± 8.4 | 61.7 ± 1.1 | 69.8 ± 1.0 | 151.8 ± 21.4 | 95.3 ± 5.2 | 69.3 ± 5.2 | 73.9 ± 1.5 |
| Mg²⁺ | 55.8 ± 9.4 | 61.8 ± 4.3 | 26.1 ± 1.7 | 69.8 ± 1.1 | 137.3 ± 8.7 | 113.2 ± 2.2 | 55.9 ± 1.9 | 73.9 ± 1.2 |
| Ca²⁺ | 25.6 ± 1.2 | 75.9 ± 6.0 | 45.1 ± 6.0 | 162.6 ± 11.2 | 0.0 ± 6.9 | 32.8 ± 1.1 | 35.8 ± 8.4 | 184.8 ± 1.3 |
| Mn²⁺ | 100.0 ± 6.0 | 8.4 ± 1.2 | 90.1 ± 1.1 | 124.1 ± 12.5 | 115.6 ± 12.5 | 68.5 ± 2.3 | 129.7 ± 20.2 | 141.1 ± 2.0 |
| Zn²⁺ | 39.5 ± 1.7 | 84.3 ± 11.9 | 7.1 ± 2.5 | 14.8 ± 2.5 | 368.6 ± 14.5 | 107.3 ± 5.0 | 107.4 ± 9.4 | 16.8 ± 1.5 |
| Co²⁺ | 32.6 ± 2.1 | 0.0 ± 10.7 | 19.0 ± 8.4 | 70.9 ± 1.3 | 57.8 ± 1.3 | 56.6 ± 1.9 | 102.9 ± 1.3 | 80.7 ± 2.0 |

Lip4 activity was strongly inhibited by NH⁴⁺, Mg²⁺, Ca²⁺, Zn²⁺ and Co²⁺ (residual activities <55%), moderately inhibited by Na⁺ and K⁺ (residual activities, 70% and 88% respectively), and no effect of Mn²⁺ was observed. Lip6 activity was totally inhibited by Mn²⁺ and Co²⁺ (residual activities, 8 and 0% respectively), and strong inhibition was also observed in the presence of K⁺, NH⁴⁺ and Mg²⁺ (residual activity <62%). Ca²⁺ and Zn²⁺ moderately inhibited lip6 activity (residual activities, 76 and 84% respectively) while Na⁺ slightly increased its activity (101%). Only Zn²⁺ had a strong inhibitory effect on pl1 activity (15% residual activity) while Na⁺, K⁺, NH⁴⁺, Mg²⁺ and Co²⁺ had moderate inhibitory effects (70% to 76% residual activities). Ca²⁺ and Mn²⁺ activated pl1 (residual activities, 163 and 124% respectively). Lip13ss activity was strongly inhibited in the presence of K⁺, Mg²⁺, Ca²⁺, Zn²⁺ and Co²⁺ (residual activities <50%) and more moderately by Na+, NH⁴⁺ and Mn²⁺ (residual activities from 61 to 90%). Pl2ss was totally inhibited in the presence of Ca²⁺ and strongly with Co²⁺ (57%), but its activity increased significantly with other ions (from 115% with Mn²⁺ to up to 368% in the presence of Zn²⁺).

Ca²⁺, Mn²⁺ and Co²⁺ inhibited alipA activity(<68% activity), K⁺ and NH⁴⁺ did not modify the activity (98-99% activity), while Na⁺, Mg²⁺ and Zn²⁺ activated alipA. AlipBss activity was strongly inhibited by Ca²⁺ and Mg²⁺ (residual activities, 33 and 56% respectively) and moderately inhibited by Na⁺, K⁺ and NH⁴⁺ (residual activities, 72, 67 and 69% respectively), whereas Mn²⁺, Zn²⁺ and Co²⁺ activated alipBss (residual activities, 130, 107, and 103% respectively). Zn²⁺ strongly inhibited alipC activity (17% residual activity); NH⁴⁺, Mg²⁺ and Co²⁺ had more moderate inhibitory effects (residual activities from 74 to 81%). K⁺ had no effect, while Na⁺, Ca²⁺ and Mn²⁺ activated alipC (residual activities, 119, 185, and 141% respectively).

Formation of Polylactic Acid Polymer (PLA)

Lactic acid polymerizing assays as described by Lassalle et al., (2008); Lassalle and Ferreira, (2008) were performed using equivalent concentrations of our rumen derived (discovered using functional metagenomics) lipases designated as Lipase B and Lipase F (Table 10; this table also provides information on our other rumen metagenomically derived lipases which have been biochemically characterised) and Novozym 435 as a positive control.

| | | | | | | | | | |
|---|---|---|---|---|---|---|---|---|---|
| Aberystwyth | | | | | | | | | |
| Lipase number | A | B | C | D | E | F | G | H | Notes |

pH

| | | | | | | | | | |
|---|---|---|---|---|---|---|---|---|---|
| Optimum | 7.5 | 7.0 | 7.5 | 7.5 | 8.5 | 9.0 | 7.5 | 9.0 | Measure |
| Range with specific | 6.5-10 | 6.0-8.0 | 6.5-8.5 | 6.5-8.0 | 8.5-10 | 7.0-10.0 | 9.0-10.0 | 9.0-10.0 | at 39° C. |
| activity >50% | | | | | | | | | |

Temperature (C. °)

| | | | | | | | | | |
|---|---|---|---|---|---|---|---|---|---|
| Optimum | 40 | 40 | 45 | 40 | 30 | 40 | 55 | 40 | Measure |
| Range with specific | 40 | 40-50 | 25-50 | 40 | 25-40 | 30-50 | 35-55 | 25-50 | at pH 6.5 |
| activity >50% | | | | | | | | | |
| Thermostability | | | | | | | | | |

Relative activity (%) after 1 h incubation at

| | | | | | | | | | |
|---|---|---|---|---|---|---|---|---|---|
| 50° C. | 57.9 | 41.8 | 37.0 | 25.7 | 87.8 | 16.8 | 33.6 | 32.8 | 100% |
| 60° C. | 11.5 | 47.5 | 15.1 | 10.5 | 89.6 | 45.3 | 24.0 | 8.2 | activity = |
| 70° C. | 13.0 | 31.3 | 16.5 | 11.4 | 73.8 | 49.2 | 15.6 | 9.8 | specific |
| | | | | | | | | | activity |
| | | | | | | | | | measured |
| | | | | | | | | | at 40° C. |

In order to isolate the newly formed polylactic acid, dichloromethane was added to the solutions, followed by dH₂O. At the interface of toluene and water, the synthesized PLA could be observed. The solutions were then frozen at -20° C. and freeze dried for 2-3 days, in order to isolate the polymer. FTIR was conducted in order to check PLA formation in comparison to Novozym 435. Freeze-dried weight was also recorded in order to assess comparative yield. Our data show that both of our lipases produced approximately 10% more PLA than novozym 435 under our test conditions and FTIR confirmed this data whilst illustrating that we had indeed made PLA. The chemical reaction in the formation of PLA is as follow:

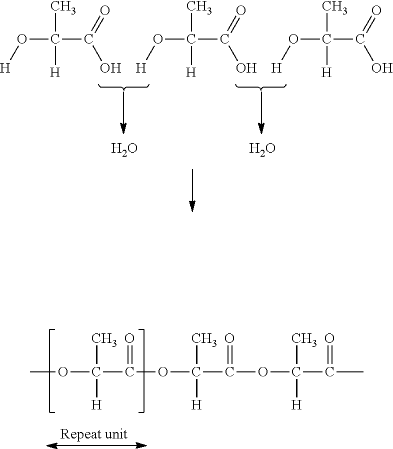

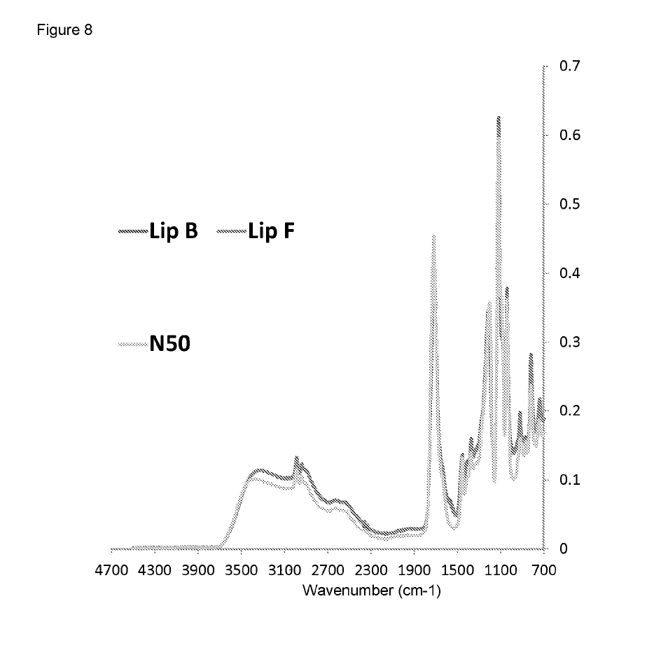

Thus the presence of C—O—C linkages increase as polymerization proceeds. Indeed in our FT-IR traces of isolated putative PLA we see characteristic ester absorption peaks at approx. 1700, 1670 for the stretching vibration of the —COO— and at 1100 and 1200 for the stretching vibration of the C—O—C, proving the production of PLA (FIG. 8).

Nucleotide Sequence Accession Numbers

The nucleotide sequences of the genes reported here are available in the Gen Bank database under accession numbers: alipA, KC579357; alipB, KC579358; alipC, KC579359; lip1, JX469447; lip2, JX469448; lip3, JX469449; lip4, JX469450; lip5, JX469451; lip6, JX469452; lip7, JX469453; lip8, JX469454; lip9, JX469455; lip10, JX469456; lip11, JX469457; lip12, JX469458; lip13, JX469459; lip14, JX469460; pl1, JX469461; and pl2, JX469462.

It will be appreciated by those skilled in the art that the foregoing description is exemplary and explanatory in nature, and is intended to illustrate the invention and its preferred embodiments. Through routine experimentation, an artisan will recognise apparent modifications and variations that may be made without departing from the spirit as scope of the invention as defined in the appended claims.

### BIBLIOGRAPHY

Abdou, A. M. (2003) Purification and partial characterization of psychrotrophic Serratia marescens lipase. J Dairy Sci 86: 127-132.
Akoh, C. C., Lee, G. C., Liaw, Y. C., Huang, T. H., and Shaw, J. F. (2004) GDSL family of serine esterases/lipases. Prog Lipid Res 43: 534-552.
Arpigny, J. L., Jaeger, K.-E. (1999) Bacterial lipolytic enzymes: classification and properties. Biochem J 343:177-183.
Aziz, R. K., Bartels, D., Best, A. A., DeJongh, M., Disz, T., Edwards, R. A., et al. (2008) The RAST Server: Rapid Annotations using Subsystems Technology. BMC Genomics 9:75.
Bakke, P., Carney, C., DeLoache, W., Gearing, M., Ingvorsen, K., Lotz, M., et al. (2009) Evaluation of three automated genome annotations for Halorhabdus utahensis. PLoS One 4(7):e6291.
Banerji, S., and Flieger, A. (2004) Patatin-like proteins: a new family of lipolytic enzymes present in bacteria? Microbiology 150: 522-525.
Bauman, D. E., Perfield, J. W. II, De Veth, M. J., and Lock, A. L. (2003) New perspectives on lipid digestion and metabolism in ruminants. Proc Cornell Nutr Conf, pp. 175-189.
Bayer, S., Kunert, A., Ballschmiter, M., and Greiner-Stoeffele, T. (2010) Indication for a new lipolytic enzyme family: isolation and characterization of two esterases from a metagenomic library. J Mol Microbiol Biotechnol 18: 181-187.
Bekele, A. Z., Koike, S., and Kobayashi, Y. (2010) Genetic diversity and diet specificity of ruminal Prevotella revealed by 16S rRNA gene-based analysis. FEMS Microbiol Lett 305: 49-57.
Bornscheuer, U. T. (2002) Microbial carboxyl esterases: classification, properties and application in biocatalysis. FEMS Microbiol Rev 26: 73-81.
Bradford, M. M. (1976) A rapid and sensitive method for the quantitation of microgram quantities of protein utilizing the principle of protein dye binding. Anal Biochem 72:248-254.
Cai., J., Xie, Y., Song, B., Wang, Y., Zhang, Z., Feng, Y. (2011) Fervidobacterium changbaicum Lip1: identification, cloning, and characterization of the thermophilic lipase as a new member of bacterial lipase family V. Appl Microbiol and Biotechnol 89:1463-1473.
Carinato, M. E., Collin-Osdoby, P., Yang, X., Knox, T. M., Conlin, C. A. and Miller, C. G. (1998) The apeE gene of Salmonella typhimurium encodes an outer membrane esterase not present in Escherichia coli. J Bacteriol 180: 3517-3521.
Chang, R. C., Chen, J. C., and Shaw, J. F. (1995) Vibrio mimicus arylesterase has thioesterase and chymotrypsin-like activity. Biochem Biophys Res Commun 213: 475-483.
Cho, H., and Cronan, J. E. Jr. (1993) Escherichia coli thioesterase I, molecular cloning and sequencing of the structural gene and identification as a periplasmic enzyme. J Biol Chem 268: 9238-9245.
Cirne, D. G., Delgado, O. D., Marichamy, S., Mattiasson, B. (2006) Clostridium lundense sp. nov., a novel anaerobic lipolytic bacterium isolated from bovine rumen. Int J Syst Evol Microbiol 56:625-628.
Dautin, N., Bernstein, H. D. (2007) Protein secretion in gram-negative bacteria via the autotransporter pathway. Annu Rev Microbiol 61:89-112.
Downie, M. M., Guy, R.,and Kealey, T. (2004) Advances in sebaceous gland research: potential new approaches to acne management. Int J Cosmet Sci 26: 291-311.
Du Plessis, E., Berger, E., Stark, T., Louw, M., and Visser, D. (2009) Characterization of a novel thermostable esterase from Thermus scotoductus SA-01: evidence of a new family of lipolytic esterases. Curr Microbiol 60: 248-253.
Enjalbert, F., and Troegeler-Meynadier, A. (2009) Biosynthesis of trans fatty acids in ruminants. In Trans fatty acids in human nutrition. Destaillats, F., Sébédio, J. L., Dionisi, F., and Chardigny, J. M. (eds). Bridgwater: The Oily Press, P. J. Barnes & Associates, pp. 1-42.
Falcocchio, S., Ruiz, C., Pastor, F. I., Saso, L. and Diaz, P. (2006) Propionibacterium acnes GehA lipase, an enzyme involved in acne development, can be successfully inhibited by defined natural substances. J Mol Catal, B: Enzym 40: 132-137.
Fay, J. P., Jakober, K. D., Cheng, K. J., Costerton, J. W. (1990) Esterase activity of pure cultures of rumen bacteria as expressed by the hydrolysis of ρ-nitrophenylpalmitate. Can J Microbiol 36:585-589.
Ferrer, M., Golyshina, O. V., Chernikova, T. N., Khachane, A. N., Martins dos Santos, V. A. P., Yakimov, M. M., et al. (2005a) Microbial enzymes mined from the Urania deep-sea hypersaline anoxic basin. Chem Biol 12: 865-904.
Ferrer, M., Golyshina, O. V., Chernikova, T. N., Khachane, A. N., Reyes-Duarte, D., Santos, V. A., et al. (2005) Novel hydrolase diversity retrieved from a metagenome library of bovine rumen microflora. Environ Microbiol 7(12):1996-2010.
Gao, F., Wang, Y., Liu, Y. J., Wu, X. M., Lv, X., Gan, Y. R., et al. (2011) Genome sequence of Acinetobacter baumannii MDR-TJ. J Bacteriol 193(9):2365-2366.
Glogauer, A., Martini, V. P., Faoro, H., Couto, G. H., Muller-Santos, M., Monteiro, R. A., et al. (2011) Identification and characterization of a new true lipase isolated through metagenomic approach. Microb Cell Fact 10: 54.
Hall, N. (2007) Advanced sequencing technologies and their wider impact in microbiology. J Exp Biol 210(9):1518-1525.
Hårdeman, F., and Sjöling, S. (2007) Metagenomic approach for the isolation of a novel low-temperature-active lipase from uncultured bacteria of marine sediment. FEMS Microbiol Ecol 59: 524-534.
Harfoot, C. G. (1978) Lipid metabolism in the rumen. Prog Lipid Res 17:21-54.
Harfoot, C. G., Hazlewood G. P. (1997) Lipid metabolism in the rumen. In The rumen microbial ecosystem. Hobson, P. N., and Stewart, C. S. (eds). Blackie Academic and Professional Publishers, pp. 382-426.
Hassing, G. S. (1971) Partial purification and some properties of a lipase from Corynebacterium acnes. Biochim Biophys Acta 242: 381-394.
Hausmann, S., Jaeger, K.-E. (2010) Lipolytic enzymes from bacteria. In Handbook of hydrocarbon and lipid microbiology. Timmis, K. N. (ed). Springer-Verlag, pp.1099-1126.
Hazlewood, G. P., Dawson, R. M. C. (1975) Isolation and properties of a phospholipid hydrolysing bacterium from ovine rumen fluid. J Gen Microbiol 89:163-174.
Hazlewood, G. P., Dawson, R. M. C. (1979) Characteristics of a lipolytic and fatty acid requiring Butyrivibrio sp. isolated from the ovine rumen. J Gen Microbiol 112:15-27.
Henderson, C. (1970) The lipases produced by Anaerovibrio lipolytica in continuous culture. Biochem J119(3):5P-6P.
Henderson, C. (1971) A study of the lipase produced by Anaerovibrio lipolytica, a rumen bacterium. J Gen Microbiol 65:81-89.
Henderson, C., Hobson, P. N., Summers, R. (1969) The production of amylase, protease and lipolytic enzymes by two species of anaerobic rumen bacteria. In Proceedings of the Fourth Symposium on Continuous Cultivation of Micro-organisms, Prague. Academic Press London, pp. 89-204.
Henderson, C., Hodgkiss, W. (1973) An electron microscopic study of Anaerovibrio lipolytica (strain 5S) and its lipolytic enzyme. J Gen Microbiol 76:389-393.
Henderson, L R., Navarro-Garcia, F., Nataro, J. P. (1998) The great escape: structure and function of the autotransporter proteins. Trends Microbiol 6:370-378.
Higaki, S., and Morohashi, M. (2003) Propionibacterium acnes lipase in seborrheic dermatitis and other skin diseases and Unsei-in. Drugs Exp Clin Res 29: 157-159.
Hobson, P. N., Mann, S. O. (1961) The isolation of glycerol fermenting and lipolytic bacteria from the rumen of the sheep. J Gen Microbiol 25:227-240.
Hobson, P. N., Summers, R. (1967) The continuous culture of anaerobic bacteria. J Gen Microbiol 47:53-65.
Hobson, P. N., Summers, R. (1966) Effect of growth rate on the lipase activity of a rumen bacterium. Nature 209:736-737.
Holt, R. A., Jones, S. J. (2008) The new paradigm of flow cell sequencing. Genome Res 18(6):839-846.
Hotta, Y., Ezaki, S., Atomi, A., Imanaka, T. (2002) Extremely stable and versatile carboxylesterase from a hyperthermophilic archaeon. Appl Environ Microbiol 68:3925-3931.
Hungate, R. E. (1966) The rumen and its microbes. Academic Press, New York, USA.
Huws, S. A., Lee, M. R., Muetzel, S. M., Scott, M. B., Wallace, R. J., Scollan, N. D. (2010) Forage type and fish oil cause shifts in rumen bacterial diversity. FEMS Microbiol Ecol 73(2):396-407.
Ingham, E., Holland, K. T., Gowland, G.,and Cunliffe, W. J. (1981) Partial purification and characterization of lipase (EC 3.1.1.3) from Propionibacterium acnes. J Gen Microbiol 124: 393-401.
Istivan, T. S., and Coloe, P. J. (2006) Phospholipase A in Gram-negative bacteria and its role in pathogenesis. Microbiology 152: 1263-1274.
Jarvis, G. N., Strompl, C., Moore, E. R. B., Thiele, J. H. (1998) Isolation and characterisation of obligately anaerobic, lipolytic bacteria from the rumen of red deer. Syst Appl Microbiol 21:135-143.
Jenkins, T. C., Wallace, R. J., Moate, P. J., and Mosley, E. E. (2008) Recent advances in biohydrogenation of unsaturated fatty acids within the rumen microbial ecosystem. J Anim Sci 86: 397-412.
JunGang, L., KeGui, Z., and WenJun, H. (2010) Cloning and biochemical characterization of a novel lipolytic gene from activated sludge metagenome, and its gene product. Microb Cell Fact 9:83.
Kalendar, R., Lee, D., Schulman, A. H. (2009) FastPCR software for PCR primer and probe design and repeat search. Genes, Genomes, Genomics 3(1):1-14.
Kalinowski, J., Bathe, B., Bartels, D., Bischoff, N., Bott, M., Burkovski, A., et al. (2003) The complete Corynebacterium glutamicum ATCC 13032 genome sequence and its impact on the production of L-aspartate-derived amino acids and vitamins. J Biotechnol 104: 5-25.
Kashima, Y., Ijima, M., Okamoto, A., Koizumi, Y., Udaka, S., Yanagida, F. (1998) Purification and characterization of intracellular esterases related to ethylacetate formation in Acetobacter pasteurianus. J Ferm Bioeng 85:584-588.
Kashima, Y., Nakajima, Y., Nakano, K., Tayama, K., Koizumi, Y., Udaka, S., et al. (1999) Cloning and regulation of ethanol-regulated esterase genes in Acetobacter pasteurianus. J Biosci Bioeng 87:9-27.
Kauppinen, S., Christgau, S., Kofod, L. V., Halkier, T., Dorreich, K., and Dalboge, H. (1995) Molecular cloning and characterization of a rhamnogalacturonan acetylesterase from Aspergillus aculeatus. J Biol Chem 270: 27172-27178.
Koike, S., Yabuki, H., Kobayashi, Y. (2007) Validation and application of real-time polymerase chain reaction assays for representative rumen bacteria. Anim Sci J78:135-141.
Kong, Y., Teather, R., and Forster, R. (2010) Composition, spatial distribution, and diversity of the bacterial communities in the rumen of cows fed different forages. FEMS Microbiol Ecol 74: 612-622.
Kouker, G., and K.-E. Jaeger (1987) Specific and sensitive plate assay for bacterial lipases. Appl Environ Microbiol 53: 211-213.
Lagesen, K., Hallin, P., Rødland, E. A., Staerfeldt, H. H., Rognes, T., Ussery, D. W. (2007) RNAmmer: consistent and rapid annotation of ribosomal RNA genes. Nucleic Acids Res 35:3100-3108.
Lassalle, V. L. and Ferreira, M. L. (2008) Lipase-catalyzed synthesis of polylactic acid: an overview of the experimental aspects. Journal of Chemical Technology and Biotechnology, 83, 11, 1493-1502
Lassalle, V., Galland, G. B. and Ferreira M. L. (2008) Lipase-catalyzed copolymerization of lactic and glycolic acid with potential as drug delivery devices. Bioprocess Biosyst Eng. 31(5), 499-508
Lee, S. W., Won, K., Lim, H. K., Kim, J. C., Choi, G. J ,and Cho, K. Y. (2004) Screening for novel lipolytic enzymes from uncultured soil microorganisms. Appl Microbiol Biotechnol 65: 720-726.
Lee, Y. L., Chen, J. C., and Shaw, J. F. (1997) The thioesterase I of Escherichia coli has arylesterase activity and shows stereospecificity for protease substrates. Biochem Biophys Res Commun 231: 452-456.
Lee, Y. P., Chung, G. H., Rhee, J. S. (1993) Purification and characterisation of Pseudomonas fluorescens SIK W1 lipase expressed in Escherichia coli. Biochim Biophys Acta 1169:156-164.
Legay-Carmier, F., and Bauchart, D. (1989) Distribution of bacteria in the rumen contents of dairy cows given a diet supplemented with soya-bean oil. Br J Nutr 61: 725-740.
Liu, K., Wang, J., Bu, D., Zhao, S., McSweeney, C., Yu, P., Li, D. (2009) Isolation and biochemical characterization of two lipases from a metagenomic library of China Holstein cow rumen. Bioche Biophys Res Commun 385:605-611.
Lourenço, M., Ramos-Morales, E., and Wallace, R. J. (2010) The role of microbes in rumen lipolysis and biohydrogenation and their manipulation. Animal 4(7): 1008-1023.
Lowe, T. M., Eddy, S. R. (1997) tRNAscan-SE: a program for improved detection of transfer RNA genes in genomic sequences. Nucleic Acids Res 25:955-964.
MacLean, D., Jones, J. D., Studholme, D. J. (2009) Application of ‘next-generation’ sequencing technologies to microbial genetics. Nat Rev Microbiol 7(4):287-296.
Madupu, R., Brinkac, L. M., Harrow, J., Wilming, L. G., Bohme, U., Lamesch, P., et al. (2010) Meeting report: a workshop on best practices in genome annotation. Database 2010 doi:10.1093/database/baq001.
Maqbool, Q. U., Johri, S., Rasool, S., Riyaz-ul-Hassan, S., Verma, V., Nargotra, A., et al. (2006) Molecular cloning of carboxylesterase gene and biochemical characterization of encoded protein from Bacillus subtilis (RRL BB1). J Biotechnol 125: 1-10.
Marchler-Bauer, A., Lu, S., Anderson, J. B., Chitsaz, F., Derbyshire, M. K., DeWeese-Scott, C., et al. (2011) CDD: a Conserved Domain Database for the functional annotation of proteins. Nucleic Acids Res 39(D):225-229.
Mardis, E. R. (2011) A decade's perspective on DNA sequencing technology. Nature 470:198-203.
Margulies, M., Egholm, M., Altman, W. E., Attiya, S., Bader, J. S., Bemben, L. A., et al. (2005) Genome sequencing in microfabricated high-density picolitre reactors. Nature 437(7057):376-380.
Marx, H., Graf, A. B., Tatto, N. E., Thallinger, G. G., Mattanovich, D., Sauer, M. (2011) Genome sequence of the ruminal bacterium Megasphaera elsdenii. Bacteriol 193(19):5578-5579.
Medini, D., Serruto, D., Parkhill, J., Relman, D. A., Donati, C., Moxon, R., et al. (2008) Microbiology in the post-genomic era. Nat Rev Microbiol 6:419-430.
Metzker, M. L. (2010) Sequencing technologies—the next generation. Nat Rev Genet 11:31-46.
Mølgaard, A. (2002) Rhamnogalacturonan acetylesterase, a member of the SGNH-hydrolase family. In Advances in Pectin and Pectinase Research. Voragen, F., Schols, H., and Visser, R. G. F. (eds). Kluwer Academic Publishers, pp. 299-313.

Mølgaard, A., Kauppinen, S., Larsen, S. (2000) Rhamnogalacturonan acetylesterase elucidates the structure and function of a new family of hydrolases. Structure 8(4):373-383.
Miskin, J. E., Farrell, A. M., Cunliffe, W. J., and Holland, K. T. (1997) Propionibacterium acnes, a resident of lipid-rich human skin, produces a 33 kDa extracellular lipase encoded by gehA. Microbiology 143: 1745-1755.
Nakamura, S., Yamada, A., Tsukagoshi, N., Udaka, S., Sasaki, T., Makino, S., et al. (1988) Nucleotide sequence and expression in Escherichia coli of the gene coding for sphingomyelinase of Bacillus cereus. FEBS J 175: 213-220.
Nam, S. H., Choi, S. H., Kang, A., Kim, D. W., Kim, R. N., Kim, A., et al. (2010) Genome sequence of Leuconostoc argentinum KCTC 3773. J Bacteriol 192(24):6094-6491.
Nam, S. H., Choi, S. H., Kang, A., Kim, D. W., Kim, R. N., Kim, A., et al. (2011) Genome sequence of Lactobacillus animalis KCTC 3501. J Bacteriol 193(5):1280-1281.
O′Toole, P. W., Austin, J. W., Trust, T. J. (1994) Identification and molecular characterization of a major ringforming surface protein from the gastric pathogen Helicobacter mustelae. Moir Microbiol 11:349-361.
Oomen, C. J., van Ulsen, P., van Gelder, P., Feijen, M., Tommassen, J., Gros, P. (2004) Structure of the translocator domain of a bacterial autotransporter. EMBO J23:1257-1266.
Petersen, T. N., Brunak, S., von Heijne, G., Nielsen, H. (2011) SignalP 4.0: discriminating signal peptides from transmembrane regions. Nature Methods 8:785-786.
Pinsirodom, P., Parkin, K. L. (2001) Lipase assays. Curr Prot Food Anal Chem C3.1.1-C3.1.13.
Pohlenz, H. D., Boidol, W., Schüttke, I.,and Streber, W. R. (1992) Purification and properties of an Arthrobacter oxydans P52 carbamate hydrolase specific for the herbicide phenmedipham and nucleotide sequence of the corresponding gene. J Bacteriol 147: 6600-6607.
Pohlner, J., Halter, R., Beyreuther, K., Meyer, T. F. (1987) Gene structure and extracellular secretion of Neisseria gonorrhoeae IgA protease. Nature 325:458-462.
Prins, R. A., Lankhorst, A., Van der Meer, P., Van Nevel, C.J. (1975) Some characteristics of Anaerovibrio lipolytica, a rumen lipolytic organism. Antonie van Leeuwenhoek 41:1-11.
Purushe, J., Fouts, D. E., Morrison, M., White, B. A., Mackie, R. I.; North American Consortium for Rumen Bacteria, et al. (2010) Comparative genome analysis of Prevotella ruminicola and Prevotella bryantii: insights into their environmental niche. Microb Ecol 60: 721-729.
Rattray, R. M., Craig, A. M. (2007) Molecular characterization of sheep ruminal enrichments that detoxify pyrrolizidine alkaloids by denaturing gradient gel electrophoresis and cloning. Microb Ecol 54:264-275.
Reynolds, R. D. (1982) Buffer-dependent variations in assay of tyrosine aminotransferase holoenzyme. Arch Biochem Biophys 219(1):140-148.
Rhee, J. K., Ahn, D. G., Kim, Y. G., and Oh, J. W. (2005) New thermophilic and thermostable esterase with sequence similarity to the hormone-sensitive lipase family, cloned from a metagenomic library. Appl Environ Microbiol 71: 817-825.
Roh, C., and Villatte, F. (2008) Isolation of a low-temperature adapted lipolytic enzyme from uncultivated micro-organism. J Appl Microbiol 105: 116-123.
Ruiz, C., Falcocchio, S., Pastor, F. I. J., Saso, L., Diaz, P. (2007) Helicobacter pylori EstV: identification, cloning and characterization of the first lipase isolated from an epsilon-proteobacterium. Appl Environ Microbiol 73(8): 2423-2431.
Schuster, S. C. (2008) Next-generation sequencing transforms today's biology. Nature Methods 5(1):16-18.
Scollan, N. D., Hocquette, J. F., Nuernberg, K., Dannenberger, D., Richardson, I., and Moloney, A. (2006) Innovations in beef production systems that enhance the nutritional and health value of beef lipids and their relationship with meat quality. Meat Sci 74(1): 17-33.
Sharma, R., Chisti, Y., and Banerjee, U. C. (2001) Production, purification, characterization and applications of lipases. Biotechnol Adv 19: 627-662.
Shendure, J., Ji, H. (2008) Next-generation DNA sequencing. Nature Biotechnol 26:1135-1145.
Sommer, P., Bormann, C., and Gotz, F. (1997) Genetic and biochemical characterization of a new extracellular lipase from Streptomyces cinnamomeus. Appl Environ Microbiol 63: 3553-3560.
Sitkiewicz, I., Stockbauer, K. E., and Musser, J. M. (2007) Secreted phospholipase A2 enzymes: better living through phospholipolysis. Trends Microbiol 15: 63-69.
St Geme, J. W. III, Cutter, D. (2000) The Haemophilus influenzae Hia adhesin is an autotransporter protein that remains uncleaved at the C terminus and fully cell associated. J Bacteriol 182:6005-6013.
Steinhauer, J., Agha, R., Pham, T., Varga, A. W., Goldberg, M. B. (1999) The unipolar Shigella surface protein IcsA is targeted directly to the bacterial old pole: IcsP cleavage of IcsA occurs over the entire bacterial surface. Mol Microbiol 32:367-377.
Stevenson, D. M., and Weimer, P. J. (2007) Dominance of Prevotella and low abundance of classical ruminal bacterial species in the bovine rumen revealed by relative quantification real-time PCR. Appl Microbiol Biotechnol 75: 165-174.
Stewart, C. S., Flint, H. J., Bryant, M. P. (1997) The rumen bacteria. In The rumen microbial ecosystem. 2^{nd} ed. Hobson, P. N., and Stewart, C. S. (eds). Blackie Academic and Professional Publishers London, pp. 10-72.
Stothard, P., Wishart, D. S. (2006) Automated bacterial genome analysis and annotation. Curr Opin Microbiol 9:505-510.
Strömpl, C., Tindall, B. J., Jarvis, G. N., Lünsdorf, H., Moore, E. R. B Hippe, H. (1999) A re-evaluation of the taxonomy of the genus Anaerovibrio, with the reclassification of Anaerovibrio glycerini as Anaerosinus glycerini gen. nov., comb. nov., and Anaerovibrio burkinabensis as Anaeroarcus burkinabensis [corrig.]. gen. nov., comb. nov. Int J Syst Bacteriol 49(4):1861-1872.
Suen, G., Stevenson, D. M., Bruce, D. C., Chertkov, 0., Copeland, A., Cheng, J. F., et al. (2011a) Complete genome of the cellulolytic ruminal bacterium Ruminococcus albus 7. J Bacteriol 193(19): 5574-5575.
Suen, G., Weimer, P. J., Stevenson, D. M., Aylward, F. 0., Boyum, J., Deneke, J., Drinkwater, C., Ivanova, N. N., et al. (2011b) The complete genome sequence of Fibrobacter succinogenes S85 reveals a cellulolytic and metabolic specialist. PLoS ONE 6(4):e18814.
Suhr, M., Benz, I., Schmidt, M. A. (1996) Processing of the AIDA-I precursor: removal of AIDAc and evidence for the outer membrane anchoring as a beta-barrel structure. Mol Microbiol 22:31-42.
Tajima, K., Aminov, R.I., Nagamine, T., Matsui, H., Nakamura, M., Benno, Y. (2001) Diet-dependent shifts in the bacterial population of the rumen revealed with real-time PCR. Appl Environ Microbiol 67:2766-2774.
Tamm, L. K., Arora, A., Kleinschmidt, J.H. (2001) Structure and assembly of beta-barrel membrane proteins. J Biol Chem 276:32399-32402.
Tamura, K., Peterson, D., Peterson, N., Stecher, G., Nei, M., and Kumar, S. (2011) MEGA5: Molecular Evolutionary Genetics Analysis using maximum likelihood, evolutionary distance, and maximum parsimony methods. Mol Biol Evol doi: 10.1093/molbev/msr121.
Thompson, J. D., Higgins, D. G., Gibson, T.J. (1994) CLUSTAL W: improving the sensitivity of progressive multiple sequence alignment through sequence weighing, position specific gap penalties and weight matrix choice. Nucleic Acids Res 22:4673-4680.
Tsutsui, K., Minami, J., Matsushita, O., Katayama, S., Taniguchi, Y., Nakamura, S., et al. (1995) Phylogenetic analysis of phospholipase C genes from Clostridium perfringens types A to E and Clostridium novyi. J Bacteriol 177: 7164-7170.
Upton, C., Buckley, J.T. (1995) A new family of lipolytic enzymes? Trends Biochem Sci 20(5): 178-179.
Vazquez-Boland, J. A., Kocks, C., Dramsi, S., Ohayon, H., Geoffroy, C., Mengaud, J., and Cossart, P. (1992) Nucleotide sequence of the lecithinase operon of Listeria monocytogenes and possible role of lecithinase in cell-to-cell spread. Infect Immun 60: 219-230.
Virk, A. P., Sharma, P., and Capalash, N. (2011) A new esterase, belonging to hormone-sensitive lipase family, cloned from Rheinheimera sp. isolated from industrial effluent. J Microbiol Biotechnol 21: 667-674.
Vujaklija, D., Schröder, W., Abramie, M., Zou, P., Le\check{s}èiae, I., Franke, P., and Pigac, J. (2002) A novel streptomycete lipase: cloning, sequencing and high-level expression of the Streptomyces rimosus GDS (L)-lipase gene. Arch Microbiol 178: 124-130.
Yang, X., Lin, X., Fan, T., Bian, J., and Huang, X. (2008) Cloning and expression of lipP, a gene encoding a cold-adapted lipase from Moritella sp.2-5-10-1. Curr Microbiol 56: 194-198.
Zhang, T., and Han, W. J. (2009) Gene cloning and characterization of a novel esterase from activated sludge metagenome. Microb Cell fact 8: 67.
Zock, J., Cantwell, C., Swartling, J., Hodges, R., Pohl, T., Sutton, K., et al. (1994) The Bacillus subtilis pnbA gene encoding ρ-nitrobenzyl esterase: cloning, sequence and high level expression in Escherichia coli. Gene 151: 37-43.

1-64. (canceled)

65. A lipase comprising a polypeptide or peptide comprising an amino acid sequence selected from the group consisting of SEQ ID NO: 1-19, or an amino acid sequence that has at least about 45%, or at least about 50%, or at least about 60%, or at least about 70%, or at least about 80%, or at least about 85%, or at least about 90%, or at least about 95%, or at least about 97%, or at least about 98%, or at least about 99% amino acid sequence identity therewith.

66. A lipase according to claim 65:
wherein the lipase comprising the polypeptide or peptide comprising one of the sequences of SEQ ID NO: 1-5 belongs to lipase family 1.7, or
wherein the lipase comprising the polypeptide or peptide comprising the sequence of SEQ ID NO: 6 belongs to lipase family II, or
wherein the lipase comprising the polypeptide or peptide comprising one of the sequences of SEQ ID NO: 7-12 belongs to lipase family VII, or
wherein the lipase comprising the polypeptide or peptide comprising one of the sequences of SEQ ID NO: 13 and 14 belongs to the HSL lipase family (family IV), or
wherein the lipase comprising the polypeptide or peptide comprising one of the sequences of SEQ ID NO: 15 and 16 belongs to the lipase family VIII, or wherein the lipase comprising the polypeptide or peptide comprising one of the sequences of SEQ ID NO: 17 and 18 belongs to the GDSL family, or
wherein the lipase comprising the polypeptide or peptide comprising the sequences of SEQ ID NO: 19 belongs to family V, or
wherein the polypeptide or peptide is an isolated lipase, or
wherein the polypeptide or peptide is derived from bovine rumen metagenome, or
wherein the polypeptide or peptide is derived from Anaerovibrio lipolytica 55.

67. A first nucleic acid molecule comprising a sequence of nucleotides which encodes a polypeptide or peptide or fragment or variant according to claim 65, or a second nucleic acid molecule comprising a sequence of nucleotides which is complementary to the first nucleic acid sequence or hybridizable to the first nucleic acid sequence under stringent conditions, wherein optionally:
wherein the first or second nucleic acid molecule is isolated, and/or
wherein the first or second nucleic acid molecule comprises a nucleic acid sequence, and/or
wherein the first or second nucleic acid molecule comprises single or double stranded DNA or RNA, and/or
wherein the first or second nucleic acid molecule further comprises vector nucleic acid sequences, and/or
wherein the first or second nucleic acid molecule further comprises nucleic acid sequences encoding a heterologous polypeptide.

68. A first or second nucleic acid molecule according to claim 67 which is within a host cell or a vector.

69. A process for the preparation of a lipase according to claim 65, the process comprising the steps of culturing an organism that expresses the lipase in a culture medium and recovering the lipase from the culture medium, wherein optionally the organism is a host cell, preferably a mammalian host cell or a non-mammalian host cell.

70. A lipase according to claim 65 obtainable by a process comprising the steps of culturing an organism that expresses the lipase in a culture medium and recovering the lipase from the culture medium, wherein optionally the expression takes place in a suitable host cell.

71. A process according to claim 69, wherein the host cell is transformed with a polynucleotide or polynucleotide fragment selected from the group consisting of SEQ ID NO: 20-38, or a polynucleotide or polynucleotide fragment with sequence that has at least about 45%, or at least about 50%, or at least about 60%, or at least about 70%, or at least about 80%, or at least about 85%, or at least about 90%, or at least about 95%, or at least about 97%, or at least about 98%, or at least about 99% nucleic acid sequence identity therewith.

72. A lipase according to claim 70, wherein the host cell is transformed with a polynucleotide or polynucleotide fragment selected from the group consisting of SEQ ID NO: 20-38, or a polynucleotide or polynucleotide fragment with sequence that has at least about 45%, or at least about 50%, or at least about 60%, or at least about 70%, or at least about 80%, or at least about 85%, or at least about 90%, or at least about 95%, or at least about 97%, or at least about 98%, or at least about 99% nucleic acid sequence identity therewith;
wherein optionally the lipase is obtainable from the expression of a sequence selected from the group of SEQ ID NO: 20-24 belongs to lipase family 1.7, or the lipase obtainable from the expression of a sequence of SEQ ID NO: 25 belongs to lipase family II, or the lipase obtainable from the expression of a sequence selected from the group of SEQ ID NO: 26-31 belongs to lipase family VII, or the lipase obtainable from the expression of a sequence selected from the group of SEQ ID NO: 32-33 belongs to the lipase family IV (HSL lipase family), or the lipase obtainable from the expression of a sequence selected from the group of SEQ ID NO: 34-35 belongs to lipase family VIII, or the lipase obtainable from the expression of a sequence selected from the group of SEQ ID NO: 35-37 belongs to the GDSL family, or the lipase obtainable from the expression of a sequence selected from the group of SEQ ID NO: 38 belongs to family V.

73. An antibody, that binds specifically with one or more lipases according to claim 65.

74. A lipase according to claim 65, wherein the lipase is immobilized on a solid carrier material, wherein optionally the carrier material is a polymer support, a resin, a macroporous resin, celite or silica gel and/or a combination thereof, and wherein said polymer support is optionally a polystyrene support or an acrylic support.

75. A process for making a substance using a lipase according to claim 65, wherein optionally the lipase is used in the preparation of a polymer, and wherein preferably the polymer is bioplastic, biocompatible, biodegradable, in the form of a film, a fibre or a nanoparticle, amorphous or crystalline, a copolymer, optionally comprising a diacid, a diol or a hydroxyl acid.

76. A process according to claim 75 comprising reacting one or more substrates in the presence of the lipase, wherein optionally:
the substrate is selected from the group of a lactide, a cyclic lactide dimmer and a low molecular weight polylactic acid oligomer, and/or
the substrate is a lactide selected from the group of an L-lactide, a D-lactide and a DL-lactide, and/or
the process is selected from the group of polymerization, ring opening polymerization reaction and an enzymatic copolymerization reaction.

77. The product of the process according to claim 75, wherein the product is a high molecular weight polymer.

78. A lipase according to claim 65 which is in the form of a formulation, wherein the formulation optionally further comprises a detergent and/or optionally comprises, in addition to the lipase, stabilizers, further detergents, enzyme substrates or combinations thereof.

79. A process comprising using a lipase according to claim 65 as a catalyst; or a process for the enzyme-catalytic conversion or enantioselective conversion of substrates comprising reacting substrates in the presence of the lipase, wherein the substrate is optionally an alcohol, amine, amino acid ester, and/or carboxylic acid ester; or a process for the preparation of optically active compounds comprising reacting stereoisomeric mixtures or racemates of a substrate in an enzyme catalyzed manner enantioselectively in the presence of the lipase, and resolving the mixture.

80. A lipase according to claim 65 which is present in a diagnostic kit, wherein the kit optionally further comprises an antibody and a probe

81. An antibody according to claim 73 which is present in a diagnostic kit.

82. A method of diagnosis comprising contacting a sample with a lipase according to claim 65 or an antibody binding to the lipase.

83. A method of preparation of biofuels, oil biodegradation, waste treatment or leather degreasing which uses the lipase according to claim 65, wherein optionally the biofuel is biodiesel.

84. A method for treating a patient, the method comprising administering to a patient a therapeutically effective amount of a peptide or polypeptide according to claim 65, and/or a first nucleic acid molecule comprising a sequence of nucleotides which encodes said polypeptide or peptide, or fragment or variant thereof, or a second nucleic acid molecule comprising a sequence of nucleotides which is complementary to the first nucleic acid sequence or hybridizable to the first nucleic acid sequence under stringent conditions, and/or an antibody that specifically binds the peptide or polypeptide;
wherein optionally the method comprising administering to a patient a therapeutically effective amount of a combination of two or more of the peptides or polypeptides, and/or a combination of two or more of the nucleic acid sequences, and/or a combination of two or more of the antibodies.

85. A peptide or polypeptide according to claim 65 in the form of a composition, wherein optionally there are two or more such peptides or polypeptides, and wherein optionally the composition is a pharmaceutical composition.

86. A nucleic acid sequence according to claim 67 in the form of a composition, wherein optionally there are two or more such sequences, and wherein optionally the composition is a pharmaceutical composition.

87. An antibody according to claim 73 in the form of a composition, wherein optionally there are two or more such antibodies, and wherein optionally the composition is a pharmaceutical composition.
